(19) United States
(12) Patent Application Publication
(10) Pub. No.: US 2015/0037372 A1
(43) Pub. Date: Feb. 5, 2015

(54) pH-Responsive High-Density Lipoprotein-Like Particle Complex

(71) Applicant: RESEARCH & BUSINESS FOUNDATION SUNGKYUNKWAN UNIVERSITY, Suwon-si (KR)

(72) Inventors: Dae Hyuk KWEON, Suwon-si (KR); Jae Yoon SHIN, Incheon (KR)

(73) Assignee: RESEARCH & BUSINESS FOUNDATION SUNGKYUNKWAN UNIVERSITY, Suwon-si (KR)

(21) Appl. No.: 14/335,263

(22) Filed: Jul. 18, 2014

Related U.S. Application Data
(63) Continuation-in-part of application No. PCT/KR2012/009460, filed on Nov. 9, 2012.

(30) Foreign Application Priority Data
Jan. 20, 2012 (KR) 10-2012-0007016
Nov. 7, 2012 (KR) 10-2012-0125518

(51) Int. Cl. A61K 47/42; A61K 31/704; A61K 39/02; A61K 31/4412; A61K 31/427; A61K 9/107; A61K 31/337; A61K 31/519

(52) U.S. Cl. CPC A61K 47/42; A61K 31/337; A61K 31/704; A61K 31/519; A61K 31/4412; A61K 31/427; A61K 9/1075; A61K 39/02
USPC 424/234.1; 514/449; 514/34; 514/249; 514/350; 514/365

(57) ABSTRACT

The present invention relates to a high-density lipoprotein (HDL)-like particle composite which is a drug-encapsulating apoA-I comprising apoA-I and a drug encapsulated in the apoA-I capable of releasing the drug encapsulated in the apoA-I under acidic pH conditions, and a preparation method thereof. Since the HDL-like particle composite of the present invention can release the encapsulated drug under acidic conditions, while remaining stable under non-acidic conditions over a wide temperature range, it can be widely used for the development of target-specific drugs.

### CROSS REFERENCE TO RELATED APPLICATIONS

This application is a continuation in part of and claims the benefit of priority from international application PCT/KR2012/009460 filed on Nov. 9, 2012, in the Korean Intellectual Property Office, the entire disclosure of which is incorporated herein by reference for all purposes.

### SEQUENCE LISTING

The instant application contains a Sequence Listing which has been submitted in ASCII format via EFS-Web and is hereby incorporated by reference in its entirety. Said ASCII copy, created on Sep. 29, 2014, is named NTFMP20140729_{—}0420920019_SequenceListingCRF.txt and is 4,531 bytes in size.

### BACKGROUND OF THE INVENTION

1. Field of the Invention

The present invention relates to a pH-responsive high-density lipoprotein-like particle composite, and more particularly, to a drug-encapsulating apoA-I capable of releasing a drug encapsulated in apolipoprotein A-I under acidic pH conditions and a preparation method thereof.

2. Description of the Prior Art

In recent years, the usefulness of liposome-based drug delivery systems has led to studies being focused on the preparation of more effective lipid-based nanocarriers by mixing various lipid components. Particularly, studies have been actively focused on the preparation of liposomes composed of various components for the highly efficient delivery of a physiologically active component encapsulated in the liposomes to be delivered to cells in vivo, thereby maximizing its efficacy. Examples of such studies include studies on liposome-type nanoparticles (NPs) that can deliver drugs under environmental conditions (pH, temperature, etc.) specific to certain types of cells in order to increase the selectivity of the drugs. Although cells in vivo generally have uniform environmental conditions (particularly, temperature conditions, etc.) they may occasionally have cell-specific or tissue-specific environmental conditions, for example, tissues with cancer show a lower pH level compared to that of normal tissues.

In order to deliver a substance or therapeutic drug to a tissue or lesion requiring specific environmental conditions as described above, the development of liposomes composed of nanoparticles that can control the release of a drug encapsulated therein under the environmental conditions is necessarily required. Examples of the liposomes developed include a liposome complex comprising poly(ethyl acrylic acid) and phosphatidylcholine, which are linked to the outside of a liposome so as to allow the structure of the liposome to vary with change in acidity (Tirrell et al., Macromolecules, 1984, 17, 1692), a liposome complex comprised of aliphatic polymers including poly(ethyl acrylic acid) so as to allow the structure of the aliphatic polymers composed of liposome to vary with a change in acidity (Francis et al., Biomacromolecules 2001, 2, 741), and a polymer-liposome nano-complex comprised of acidity-sensitive polymers composed of methacrylic acid and n-alkyl methacrylate (Korean Patent No. 716802). However, it was found that the aliphatic polymer including acrylic acid has a disadvantage in that it cannot form a stable liposome due to the rigidity of acrylic acid.

In order to overcome this disadvantage, various studies have been conducted. As a result, a liposome-drug complex prepared from a peptide-phospholipid particle composed of a flexible phospholipid, an unsaturated fatty acid and a peptide (WO 2009/073984), a pH-responsive liposome composed of a cationic amphiphilic molecule and an anionic amphiphilic molecule (WO 2010/104128), and the like were developed. The developed drug complex or liposome is configured to comprise multiple components that impart various characteristics, but the drug complex or liposome comprising multiple components as described above has a disadvantage in that the long-term stability thereof has not been proven. A medicine that is used for the treatment of a specific disease is not prepared immediately after its occurrence in the patient, but rather is delivered to the patient in need of administration after advance preparation at a large scale followed by storage. Accordingly, for the medicinal application of the above-described liposome, the long-term stability of the liposome should be proven. However, the developed drug composites or liposomes have not demonstrated the required long-term stability and have low resistance to various salts due to various components contained therein. Accordingly, there is a need to develop a drug complex or liposome using the material capable of controlling the release of the drug depending on a tissue-specific change in pH while, at the same time, maintaining a long-term stability.

Under this background, the present inventors have found that, when a drug-encapsulating apolipoprotein A-I (apoA-I) is formed using apoA-I, which is known as a lipoprotein, it can control the release of the drug therefrom in a pH-dependent manner and, at the same time, maintain long-term stability due to being composed of a single material, thereby completing the present invention.

### SUMMARY OF THE INVENTION

It is an object of the present invention to provide a drug-encapsulating apoA-I comprising apoA-I and a drug encapsulated therein capable of releasing the drug encapsulated in the apoA-I under acidic pH conditions.

Another object of the present invention is to provide a method for preparing the drug-encapsulating apoA-I.

To achieve the above objects, in one aspect, the present invention provides a drug-encapsulating apoA-I comprising apoA-I and a drug encapsulated in the apoA-I capable of releasing the drug encapsulated in the apoA-I under acidic pH conditions.

As used herein, the term “apoA-I (apolipoprotein A-I)” refers to a protein which is composed of a single polypeptide having a molecular weight of 28 kDa and consisting of 243 amino acids and which has eight repeat unit domains, each consisting of 11 amino acids or 22 amino acids, and in which the content of secondary alpha-helices that form high-density lipoproteins (HDL) is 60-75%. In an embodiment, apoA-I used in the present invention may be either a protein comprising an amino acid sequence of SEQ ID NO: 1 or a protein that may be expressed from a polynucleotide sequence of SEQ ID NO: 2. In another embodiment, apoA-I may be used as a component of a high-density lipoprotein (HDL) that mainly serves to remove cholesterol from the surrounding tissues and transport it to the liver or other high-density lipoprotein (HDL). For the purpose of the present invention, apoA-I may be any wild-type or mutant protein capable of functioning to encapsulate a desired drug therein and to release the encapsulated drug in a pH-dependent manner. For example, apoA-I that is used in the present invention may be a wild-type apoA-I, or a protein mutant or an artificial variant thereof, which has an amino acid sequence comprising a substitution, deletion, insertion or addition of one or more amino acids at one or more positions of the amino acid sequence of the wild-type apoA-I, as long as it can encapsulate a desired drug therein and release the encapsulated drug in a pH-dependent manner. In addition, apoA-I used in the present invention may also be a protein having a homology of at least 80%, preferably at least 90%, more preferably at least 95%, and even more preferably at least 97%, to the amino acid sequence of the wild-type apoA-I, but is not particularly limited thereto.

As used herein, the term “drug” refers to a substance that can be encapsulated in apoA-I and released from the apoA-I under acidic pH conditions to exhibit its effect. For the purpose of the present invention, the drug may be any substance that may be administered to a tissue having an acidic pH to treat, alleviate or prevent a specific disease or disorder, but is not particularly limited thereto. Preferred examples of the drug used in the present invention include, but are not limited to, paclitaxel, doxorubicin, methotrexate (MIX), Sorafenib (SF), Epothilone B (EpoB), cyclosporine, ursodeoxycholic acid (UDCA), Ilaprazole, and amine derivatives (e.g., leevec, etc.), wherein a part of the drug comprises a ring structure, and thus are easily encapsulated into apoA-I and released from apoA-I, as well as drugs having a steroidal backbone.

In an example of the present invention, PTX-PL-NP (i.e., a drug-encapsulating apoA-I), which is composed of phospholipid, paclitaxel (PTX) and apoA-I and can release the encapsulated drug in a pH-dependent manner, was prepared by emulsifying the drug paclitaxel (PTX) by heating in the presence of phospholipid and a surfactant, adding and mixing apoA-I with the emulsion while reducing the concentration of the surfactant to its critical micelle concentration or lower by dilution, and subjecting the mixture to centrifugation and size exclusion chromatography (Example 1-2). In another example of the present invention, a drug-encapsulating apoA-I (PTX-NP) composed of PTX and apoA-I was prepared by sonicating the drug paclitaxel (PTX) in the presence of a surfactant to encapsulate PTX in micelles composed of the surfactant, adding and mixing apoA-I with the micelles while reducing the concentration of the surfactant to its critical micelle concentration or lower by dilution, and subjecting the mixture to centrifugation and size exclusion chromatography (Example 1-3). In addition, the yields of the prepared PTX-PL-NP and PTX-NP were measured to be 66% and 62.2%, respectively (Example 2-1 and Tables 1 and 2).

In addition, the size and shape of each of the prepared PTX-PL-NP and PTX-NP that are encapsulated by apoA-I were analyzed, and as a result, it was found that PTX-PN-NP had a diameter of 12.2 nm, whereas PTX-NP had a diameter of 9.2 nm (FIGS. 2c and 2d). Also, it was found that PTX-NP had a spherical shape (FIG. 3), whereas PTX-PL-NP showed a ring-like structure having a halo-like circle at the rim (FIGS. 3c and 3d).

Moreover, the pH-dependent release of PTX from each of the prepared PTX-PL-NP and PTX-NP that are encapsulated by apoA-I was examined, and as a result, it was found that PTX-PL-NP was structurally stable at a pH of 3.8-10.0, whereas PTX-NP was structurally stable at neutral and basic conditions (pH 7.7-10.0), but was structurally disassembled under acidic conditions (pH 3.8 and 5.5) to release the encapsulated PTX (FIG. 4a). In addition, it was shown that PTX-NP was structurally stable even in a wide temperature range under neutral and basic conditions (FIG. 5).

Meanwhile, in order to examine whether drug-encapsulating apoA-I can be prepared when drugs other than PTX are used, an drug-encapsulating apoA-I was prepared by adding apoA-I to each of doxorubicin (DX), methotrexate (MTX), Sorafenib (SF) and Epothilone B (EpoB), and the sizes thereof were measured. As a result, it could be seen that the sizes of the prepared drug-encapsulating apoA-I had no significant difference from that of apoA-I not treated with any drug, suggesting that these drugs can also be encapsulated into apoA-I, similar to PTX (FIG. 6). In addition, EpoB released from a drug-encapsulating apoA-I (Epo-NP) comprising the drug EpoB was compared to the original EpoB, and as a result, it was found that the released drug EpoB did not chemically change (FIG. 7). Also, it was shown that Epo-NP can release the encapsulated EpoB in a pH-dependent manner (FIG. 8). Furthermore, it was shown that, when ovarian SK-OV3 cells, which overexpress HER2 and express Sr-BI receptor at a low level, were treated with varying concentrations of a drug or a drug-encapsulating apoA-I comprising the drug, treatment with the drug caused a high level of damage to the SK-OV3 cells, whereas treatment with the drug-encapsulating apoA-I comprising the drug caused a low level of damage to the SK-OV3 cells (FIG. 9).

In another aspect, the present invention provides a method for preparing the drug-encapsulating apoA-I. Specifically, the method for preparing the drug-encapsulating apoA-I according to the present invention comprises: (a) sonicating a buffer comprising a desired drug and a surfactant to encapsulate the drug into micelles composed of the surfactant, thereby obtaining a sonicated composition; (b) diluting the sonicated composition by adding a buffer comprising apoA-I to the sonicated composition, thereby obtaining a diluted composition; and (c) recovering the drug-encapsulating apoA-I from the diluted composition. The apoA-I used in the present invention may be apoA-I purified by partially removing endotoxin. In an embodiment, apoA-I used in the present invention may contain 1-1000 EU/mg of endotoxin. In another embodiment, apoA-I used in the present invention may contain 1-100 EU/mg of endotoxin.

In the preparation method of the present invention, the drug may be a drug that comprises a ring structure as a part, and thus is easily encapsulated in apoA-I and released from apoA-I. Preferably, it may be paclitaxel, but is not limited thereto. Further, the surfactant used in the present invention may be a surfactant capable of forming micelles by sonication and may preferably be sodium cholate, but is not particularly limited thereto. In addition, the buffer that is used in the present invention may be any buffer that can be maintained at a neutral pH. Preferably, the buffer may be a disc formation buffer (pH 7.4) containing 10 mM Tris HCl and 100 mM NaCl, but is not particularly limited thereto. Meanwhile, sonication in the method of the present invention may be performed by radiating sound waves at short time intervals under minimum output conditions at a low temperature in such a manner that no heat is generated. Preferably, sonication may be performed by radiating sound waves 1-10 times for 5-20 seconds at intervals of 1-3 seconds with an output of 5-20% of the total output at a temperature of 5˜15° C., but is not particularly limited thereto.

Meanwhile, dilution in the preparation method of the present invention is performed for the purpose of lowering the concentration of the surfactant in the reaction solution to remove micelles formed by the surfactant and the purpose of adding apoA-I to the drug in a suitable molar ratio. Generally, the dilution is performed by adding the same buffer containing a suitable concentration of apoA-I dissolved therein. Herein, the mixing ratio between the drug and the added apoA-I may preferably be 1:10 to 1:200 (molar ratio), more preferably 1:10 to 1:150 (molar ratio), and most preferably 1:10 to 1:100 (molar ratio), but is not specifically limited thereto. Finally, recovery of the drug-encapsulating apoA-I may be performed by size exclusion chromatography, but is not particularly limited thereto.

Meanwhile, in order to increase the yield of the drug-encapsulating apoA-I and easily remove impurities, the preparation method of the present invention may further comprise, after step (b), a step of removing an aggregate from the diluted composition by centrifugation before completely removing the surfactant or before recovering the drug-encapsulating apoA-I from the diluted composition. Herein, removal of the surfactant may be performed by treatment with a surfactant adsorbent, dialysis, chromatography or the like. Examples of the surfactant adsorbent that may be used in the present invention include, but are not limited to, polystylene copolymers, preferably Bio-bead SM2, XAD 1, XAD 2, XAD 3, XAD 4, IRC, etc. In addition, removal of the surfactant may also be performed by chromatography such as gel filtration chromatography, but is not particularly limited thereto.

### BRIEF DESCRIPTION OF THE DRAWINGS

FIG. 1 schematically shows nanoparticles capable of releasing a substance encapsulated therein in a pH-dependent manner according to the present invention.

FIG. 2 is a set of graphic diagrams showing a process for determining the sizes of PTX-NP and PTX-PL-NP. Specifically, FIG. 2a is a graphic diagram showing the results of size exclusion chromatography analysis of PTX-NP and PTX-PL-NP; FIG. 2b is a graphic diagram showing the results of size exclusion chromatography analysis of free apoA-I; FIG. 2c is a graphic diagram showing the results of dynamic light scattering analysis of PTX-NP; and FIG. 2d is a graphic diagram showing the results of dynamic light scattering analysis of PTX-PL-NP.

FIG. 3 is a set of transmission electron microscope images of nanoparticles capable of releasing a substance encapsulated therein in a pH-dependent manner according to the present invention. Specifically, FIGS. 3a and 3b show two representative images of PTX-NP, and FIGS. 3c and 3d show two representative images of PTX-PL-NP. The scale bars represents 20 nm.

FIG. 4 is a set of graphic diagrams showing the time- and pH-dependent release of PTX from PTX-NP. Specifically, FIG. 4a shows the results obtained by measuring static light scattering, which is proportional to the amount of soluble NPs, as a function of time under various pH conditions, and FIG. 4b shows the results obtained by analyzing the uncoating of the apoA-I from PTX-NP using size exclusion chromatography (SEC).

FIG. 5 is a graphic diagram showing the thermal stability of PTX-NP. Samples were incubated for 120 hours at 4° C. (), 25° C. (∘) and 37° C. (▾). As a control of liposome, large unilamellar vesicles were incubated for 120 hours at 4° C. (Δ). Light scattering intensity was measured after removing large aggregates by centrifugation (15,000 rpm for 30 minutes).

FIG. 6 shows the results of size exclusion chromatography analysis. Specifically, FIG. 6a shows analysis results for free apoA-I, and FIG. 6b shows analysis results for an EpoB/apoA-I complex prepared according to Example 3-1.

FIG. 7 shows the results of size exclusion chromatography analysis of a complex prepared according to Example 3-2.

FIGS. 8a to 8c show the determined size of an EpoB-rHDL prepared according to Example 3-3.

FIG. 9 shows the results obtained by analyzing the size of an EpoB-rHDL, prepared according to Example 3-4, by size exclusion chromatography (FIG. 9a) and dynamic laser light scattering (FIG. 9b).

FIG. 10 shows the results obtained by analyzing the size of an EpoB-rHDL, prepared according to Example 3-5, by size exclusion chromatography (FIG. 10a) and dynamic laser light scattering (FIG. 10b).

FIG. 11 shows the results obtained by analyzing the size of an EpoB-rHDL, prepared according to Example 3-6, by size exclusion chromatography (FIG. 11a) and dynamic laser light scattering (FIG. 11b).

FIGS. 12a to 12d show the determined size of an EpoB-encapsulating apoA-I (hereinafter referred to as EpoB-rHDLs) prepared according to Example 4-1 or 4-2.

FIGS. 13a and 13b show the time- and pH-dependent release of EpoB from an EpoB-encapsulating apoA-I.

FIG. 14 is a set of graphic diagrams showing a comparison of size between apoA-I and a drug-encapsulating apoA-I prepared by reacting apoA-I with doxorubicin (DX), methotrexate (MTX), Sorafenib (SF) or Epothilone B (EpoB).

FIG. 15 is a graphic diagram showing a comparison of size between an original EpoB and an EpoB released from a drug-encapsulating apoA-I (Epo-NP) comprising EpoB.

FIG. 16 is a graphic diagram showing the relative scattering intensity of a drug-encapsulating apoA-I (Epo-NP) comprising EpoB at various pH conditions.

FIG. 17 is a graphic diagram showing the change in viability of SK-OV3 cells, treated with a drug or a drug-encapsulating apoA-I comprising the drug, as a function of the concentration of the drug or the drug-encapsulating apoA-I.

FIG. 18 shows the expression levels of SR-B1 in five kinds of cancer cell lines (MCF7, MDA-MB-231, SK-OV-3, Caco-2 and ZR-75-1).

FIG. 19 shows the viabilities of five types of cancer cells treated with an EpoB-encapsulating apoA-I.

FIG. 20 shows the viability of cancer cells, treated with an EpoB-encapsulating apoA-I, as a function of the expression level of SR-B1.

FIG. 21 shows the survival rate of ICR mice administered with test materials (PEG 300, apoA-I, EpoB-encapsulating apoA-I, and EpoB).

FIG. 22 shows changes in the body weight of ICR mice administered with test materials.

FIG. 23 shows the survival rate of ICR mice administered with test materials.

FIG. 24 shows a change in the body weight of each of ICR mice administered with test materials.

FIG. 25 shows the average body weight of ICR mice administered with each of test materials.

FIG. 26 shows the average body weight of ICR mice administered with test materials.

FIG. 27 shows percent changes in the body weight of each of ICR mice administered with test materials.

FIG. 28 shows percent changes in the average body weight of ICR mice administered with each test material.

FIG. 29 shows percent changes in the average body weight of ICR mice administered with test materials.

FIG. 30 shows whether gastric hypersensitivity caused by abdominal lymphadenopathy (i.e., the side effect of EpoB) occurred in ICR mice administered with test materials.

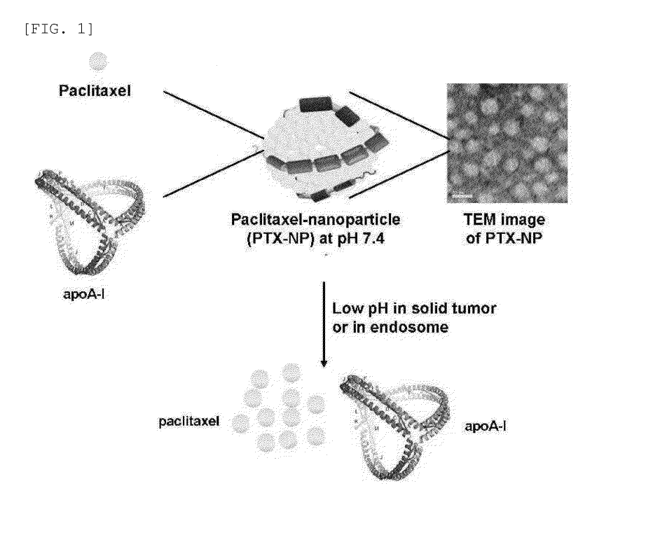

### DETAILED DESCRIPTION OF THE INVENTION

Hereinafter, the present invention will be described in further detail with reference to examples. It is to be understood, however, that these examples are for illustrative purposes only and are not intended to limit the scope of the present invention.

### EXAMPLE 1

### Preparation of Paclitaxel-Encapsulating apoA-I

### EXAMPLE 1-1

### Expression and Purification of Recombinant apoA-I

The expression of codon-optimized human apoA-I was performed using the pET protein expression system (Novagen, Madison, Wis.), with the previously described expression vector pNFXex-apoA-I (R. O. Ryan et al., Protein Expr. Purif., 27:98-103, 2003). Purification of His-tagged apoA-I was performed as previously described (J. Y. Shin et al., Biochem. Biophys. Res. Commun., 388:217-221, 2009).

### EXAMPLE 1-2

### Preparation of Drug-Encapsulating apoA-I (PTX-PL-NP) using Phospholipid

1-Palmitoyl-2-oleoyl-sn-glycero-3-phosphocholine (POPC, Avanti Polar Lipids, Alabaster, Ala.), a kind of phospholipid, was prepared as a 25 mM stock solution in chloroform, and was dried under a stream of nitrogen gas to produce a lipid film on the inner wall of a test tube. The glass tube containing the phospholipid film was placed in vacuum overnight to remove the residual solvent. The film of dried POPC was then dissolved by heating at 60° C. in disc formation buffer (10 mM Tris HCl, 100 mM NaCl, pH 7.4) supplemented with 100 mM sodium cholate, a kind of surfactant, resulting in a final lipid to cholate ratio of 1:4, and was further dissolved at 37° C. for 2 hours to produce full dissolution. Then, 0.1 mL of 1.64 mM POPC solution was mixed with 1.4 mg/mL (1.64 mM) of paclitaxel (PTX), which was dissolved in the disc formation buffer containing 100 mM sodium cholate. The concentration of sodium cholate was lowered below its critical micelle concentration by diluting the mixture solution with 2 mL of the disc formation buffer containing purified apoA-I. In the final mixture, the molar ratio of apoA-I to (PTX+POPC) was maintained at 1:75.

The diluted mixture solution was centrifuged at 15,000 rpm for 30 minutes to remove the protein/lipid precipitate, and the supernatant was collected. The supernatant was subjected to size exclusion chromatography (SEC) using a Superdex-200 10/300 GL column on an ACTA FPLC apparatus (GE Healthcare, Buckinghamshire, UK) while the temperature was maintained at 25° C. Herein, the column was equilibrated using suitable buffer (10 mM Tris HCl, pH 8.0), and then 100 μL of the sample was injected into the column at a flow rate of 0.5 mL/min.

The results of size exclusion chromatography (SEC) indicated that PTX-PL-NP, i.e., a drug-encapsulating apoA-I, was prepared and that this procedure resulted in a final PTX recovery yield of 50%-60%.

### EXAMPLE 1-3

### Preparation of Drug-Encapsulating apoA-I (PTX-NP) Without Using Phospholipid

PTX-NP, i.e., a drug-encapsulating apoA-I comprising no phospholipid, could not be formed by the method described in Example 1-2.

Thus, in order to develop a method capable of preparing PTX-NP in high yield without using a phospholipid, a disc formation buffer containing 100 mM sodium cholate and 1.4 mg/mL (1.64 mM) PTX was sonicated to encapsulate PTX in micelles formed by sodium cholate. Herein, the sonication was repeated three times at 1-second intervals for a total of 10 seconds at low temperature with a minimum output of 20% or less using a micro-tip probe sonicator having a size capable of penetrating the sample, in such a manner that no heat generation occurred. Next, the sonicated disc formation buffer was diluted with 2 mL disc formation buffer containing apoA-I to lower the concentration of sodium cholate below its critical micelle concentration. Herein, the mixing ratio of apoA-I to PTX was 1:100 (molar ratio).

Thereafter, the surfactant adsorbent Bio-beads SM-2 (bio-Rad Laboratories) was added to the diluted mixture solution to completely remove sodium cholate present at its critical micelle concentration or lower, and was sequentially subjected to centrifugation and size exclusion chromatography (SEC) in the same manner as described in Example 1-2, thereby preparing PTX-NP. The concentrations of apoA-I and PTX in the prepared PTX-NP and the yield of the prepared PTX-NP were measured.

### EXAMPLE 1-4

### Size Distribution

The size distribution of PTX-PL-NPs and PTX-NPs that are contained in the drug-encapsulating apoA-I prepared in Examples 1-2 and 1-3, respectively, was determined by a dynamic laser light scattering technique using a Dynapro apparatus (Wyatt Technology, Santa Barbara, Calif.). The scattering angle and temperature were fixed at 90° C. and 25° C., respectively. Energy filtered-transmission electron microscopy was performed using a LIBRA 120 electron microscope (Carl Zeiss, Jena, Germany). After deposition of the drug-encapsulating apoA-I onto a 100 mesh copper grid coated with carbon, the grid was tapped to a filter paper to remove surface water and negatively stained using 2% uranyl acetate. The samples were air-dried before measuring.

### EXAMPLE 1-5

### Quantitative Assay of Paclitaxel

Paclitaxel (PTX) was quantified using a high-pressure liquid chromatography apparatus equipped with a Capcell Pak C18 MG reversed-phase column (Shiseido, Tokyo, Japan) and an ultraviolet detector. Pure ethanol was used as an isocratic mobile phase at a flow rate of 1.0 mL/minute. The concentration of paclitaxel was quantified from a standard curve.

### EXAMPLE 1-6

### Time-Dependent Uncoating of apoA-I from Drug-Encapsulating apoA-I

Release of PTX from drug-encapsulating apoA-I was observed via static light scattering assay. Static light scattering was measured using a Molecular Device SpectraMax M2 Fluorimeter at an excitation and emission wavelength of 500 nm. After reading the value for 2 hours at the same pH (7.4), the pH values of the samples were lowered or elevated.

### EXAMPLE 2

### Results (Paclitaxel-Encapsulating apoA-I)

### EXAMPLE 2-1

### Preparation of Drug-Encapsulating apoA-I

Various drug-encapsulating HDL-like nanoparticles reported to date do not exhibit pH-responsiveness. Also, nearly all HDL-like NPs contained phospholipids, cholesterol and cholesteryl ester as well as the lipophilic drug of interest. Because phospholipids such as phosphatidylcholine are important to apoA-I binding, which encompasses the edge of the discoidal nanostructure, the present inventors hypothesized that exclusion of phospholipids from the nanoparticle might result in sensitivity to environmental changes, such as pH. Also, the initial concentration of paclitaxel used in the preparation of each of the composites, the final concentration of paclitaxel encapsulated in each of the composites, and the yield of the prepared composites were calculated.

In the case of PTX-PL-NP prepared by the method of Example 1-2, the initial concentration of paclitaxel and the concentration of paclitaxel encapsulated into the composite were quantitatively analyzed by reverse-phase chromatography. Based on the analysis results, each chromatographic area was calculated, and the ratio of the area of paclitaxel encapsulated into each composite to the area of initial paclitaxel was calculated, thereby determining the final concentration and yield of paclitaxel encapsulated into each composite (Table 1).

TABLE 1
Concentration and yield of paclitaxel contained in PTX-PL-NP

| | Initial | Final | |
|---|---|---|---|
| | paclitaxel | paclitaxel | Yield |
| Preparation method | (μg/mL) | (μg/mL) | (%) |
| Dilution | 1400 | ND | ND |
| Dialysis | 1400 | ND | ND |
| Heating + dilution | 1400 | 302 | 22 |
| Sonication + dilution | 1400 | 924 | 66 |

^{a}The concentration of paclitaxel dissolved in the mixture solution before detergent removal.
^{b}The concentration of paclitaxel present in drug-encapsulating apoA-I after SEC purification.
ND: Not detected.

As shown in Table 1 above, even though apoA-I is highly soluble in aqueous medium, it could be seen that, when dilution and dialysis were used alone, PTX-encapsulating normal apoA-I was not formed, but when heating and dilution were sequentially used or when sonication and dilution were sequentially used, PTX-encapsulating normal apoA-I was formed.

Particularly, when sonication was used instead of heating, the yield increased by about 3 times, suggesting that, when sonication is used instead of heating, the efficiency with which PTX is emulsified can be increased, thereby increasing the yield of the drug-encapsulating apoA-I.

In the case of PTX-NP prepared according to the method of Example 1-3 without using phospholipid, unlike PTX-PL-NP, it was shown that, when simple dilution and dialysis were used or when heating and dilution were sequentially used, PTX-encapsulating normal apoA-I was not formed, suggesting that a drug-encapsulating apoA-I can be prepared by sequentially using sonication and dilution (Table 2).

TABLE 2
Concentration and yield of apoA-I and paclitaxel contained in PTX-NP

| Preparation | apoA-I | Final paclitaxel | |
|---|---|---|---|
| method | (μg/mL) | (μg/mL) | Yield (%) |
| Sonication + | 560 | 924 | 62.2 |
| dilution | | | |

As shown in Table 2 above, the yield of the drug-encapsulating apoA-I prepared by sequentially subjecting to sonication and dilution was 62.2%, which was similar to the yield of the composite prepared according to the method of Example 1-2. In addition, the molar ratio of apoA-I to paclitaxel in the prepared PTX-NP was about 1:55, suggesting that about 110 paclitaxel (PTX) molecules were included in each apoA-I molecule.

### EXAMPLE 2-2

### Size and Shape of Drug-Encapsulating apoA-I

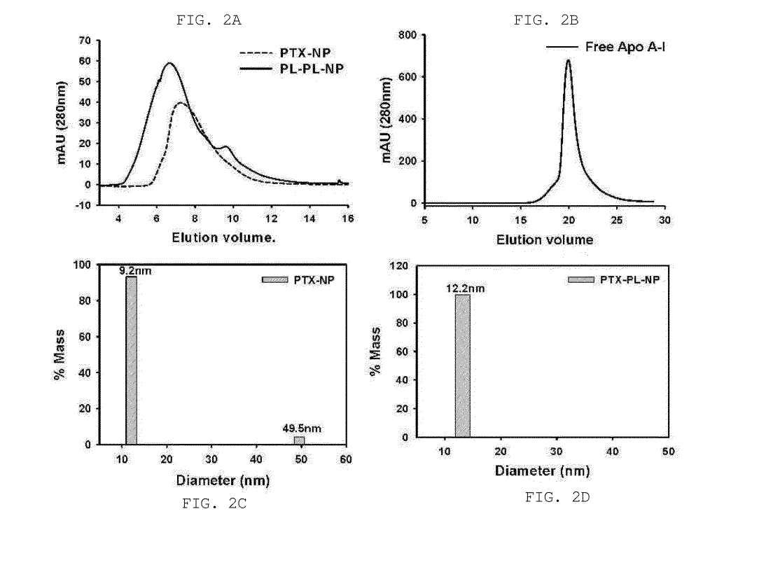

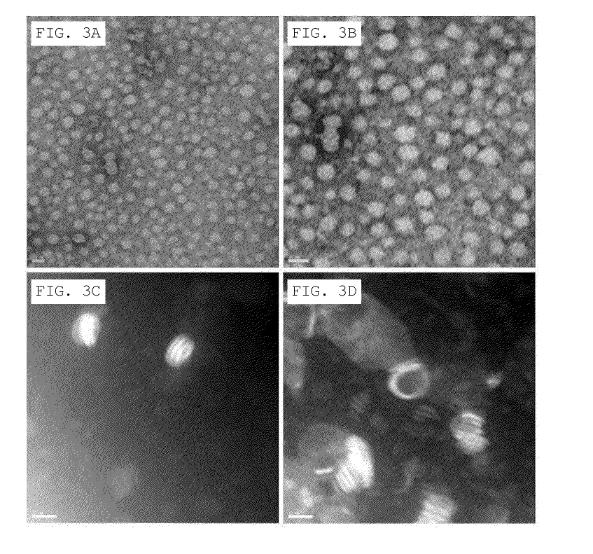

The prepared PTX-NP and PTX-PL-NP that are drug-encapsulating apoA-I were analyzed by size exclusion chromatography, and as a result, it was found that the peak of PTX-NP was slightly shifted to an elution volume higher than that of PTX-PL-NP, indicating that PTX-NP has a size smaller than PTX-PL-NP (FIG. 2a).

In addition, the prepared PTX-NP and PTX-PL-NP were subjected to dynamic light scattering analysis, and as a result, it was shown that PTX-PN-NP had a diameter of 12.2 nm, whereas PTX-NP had a diameter of 9.2 nm (FIGS. 2c and 2d).

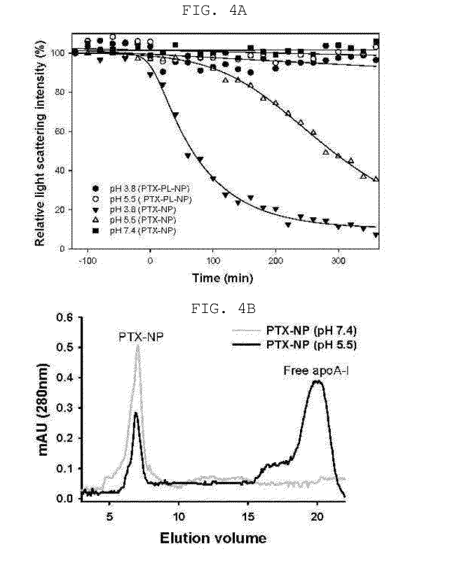

Meanwhile, the results of transmission electron microscope analysis indicated that PTX-NP had a spherical shape (FIG. 3), whereas PTX-PL-NP showed a ring shape having a halo-like circle at the rim (FIGS. 3c and 3d).

As described above, it could be seen that the prepared PTX-NP and PTX-PL-NP that are encapsulated by apoA-I showed different structural characteristics. This structural difference was likely due to the absence of phospholipids, and thus it was hypothesized that phospholipids stabilized the discoidal structure of the drug-encapsulating apoA-I.

### EXAMPLE 2-3

### pH-Responsiveness of Drug-Encapsulating apoA-I

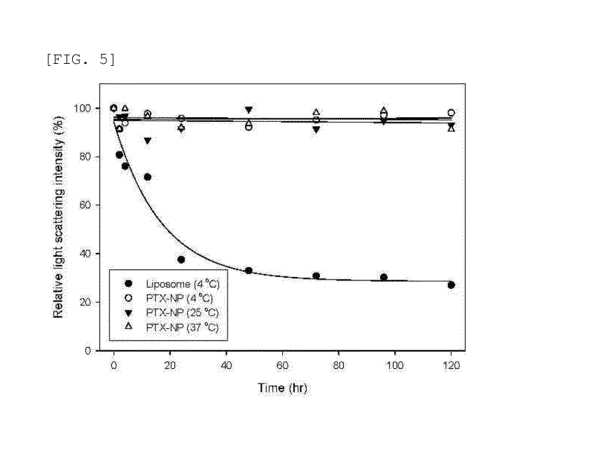

An essential feature of drug-encapsulating apoA-I for cancer chemotherapy is a selective drug release at low pH, with a stable retention of the drug inside the composite at physiological pH. To measure the pH-dependent behavior of the drug-encapsulating apoA-I, a static light scattering assay was employed. Because the size of free apoA-I is much smaller than that of the drug-encapsulating apoA-I (FIG. 2b), the release of PTX from the drug-encapsulating apoA-I can be easily detected based on a lowered static light scattering. Light scattering intensity is proportional to the amount of the drug-encapsulating apoA-I. Static light scattering of the PTX-PL-NP and PTX-NP prepared in the Examples above was measured for 6 hours at pH 3.8, 5.5 and 7.4 (FIG. 4a).

At physiological pH of pH 7.7, there was no change at all in light scattering intensity for both NPs. Although PTX-NP has no phospholipid, it was as stable as PTX-PL-NP at physiological pH. In contrast, the light scattering intensity of PTX-NP dramatically decreased at pH 5.5, and the rate of decrease in the intensity was much faster at pH 3.8. PTX-PL-NP was stably maintained even at pH 3.8. Interestingly, both NPs exhibited no significant change in light scattering intensity in the basic pH range (pH 8.2-10.0).

In order to examine whether the lowered light scattering intensity of PTX-NP at low pH was due to release of PTX, PTX-NP was incubated at pH 7.4 or 5.5 for 5 hours and analyzed by SEC. As a result, it was shown that, at physiological pH (pH 7.4), the content of PTX-NP was stably maintained, but at pH 5.5, the concentration of PTX-NP decreased and free apoA-I was generated in the solution. This result demonstrates that PTX-NP can selectively release PTX at low pH (a pH-dependent release ability), but PTX-PL-NP showed no pH-dependent release ability.

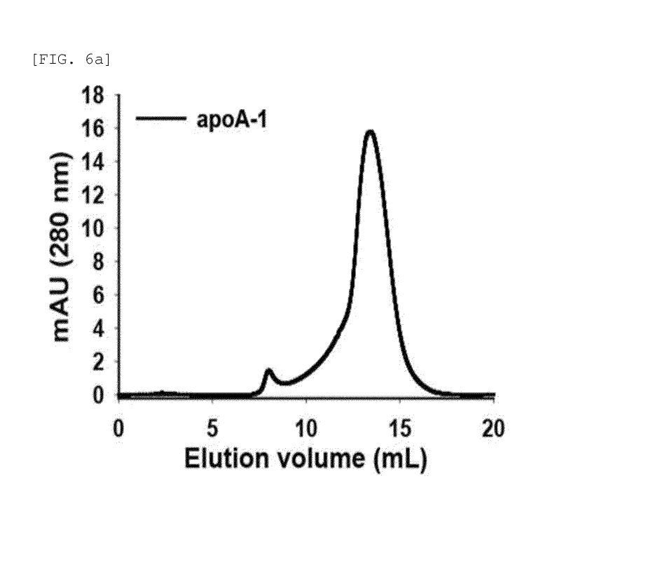

The stability of PTX-NP that indicates the pH-dependent release ability was analyzed, and as a result, it was shown that even when PTX-NP was maintained at pH 7 to 10 and a temperature of 25° C. for 5 days, the light scattering intensity did not change, and the stability thereof was maintained even in a wide temperature range (4, 25 and 37° C.), but the control composite did not show this stability (FIG. 5).

Taken together, the above results indicate that that the phospholipid-deficient PTX-NP can release the encapsulated drug in a pH-dependent manner only in an acidic pH range, suggesting that it can be used as a suitable therapeutic agent for the treatment of cancer cells in vivo, which show an acidic pH.

### EXAMPLE 3

### Trial and Error for Preparation of EpoB-Encapsulating apoA-I

### EXAMPLE 3-1

### Condition 1

A solution containing 507.68 μg of EpoB and 1 mL of 100 mM sodium cholate was sonicated to encapsulate EpoB in micelles formed by sodium cholate. The sonication was performed at 1-second intervals for a total of 1 minute with a minimum output of 20% or less at low temperature in such a manner that no heat generation occurred.

Then, the sonicated solution containing EpoB micelles was mixed with 1 mL of phosphate buffer containing apoA-I and was allowed to react at room temperature for 1 hour. Then, the mixture solution was diluted with phosphate buffer so that the concentration of sodium cholate was lowered below its critical micelle concentration while apoA-I was mixed with EpoB micelles. Herein, the ratio of apoA-I to EpoB was 1:10 (molar ratio).

Next, a surfactant adsorbent was added to the diluted mixture solution to completely remove sodium cholate present at the critical micelle concentration, and then the mixture solution was subjected sequentially to centrifugation and size exclusion chromatography, thereby preparing an EpoB/apoA-I composite.

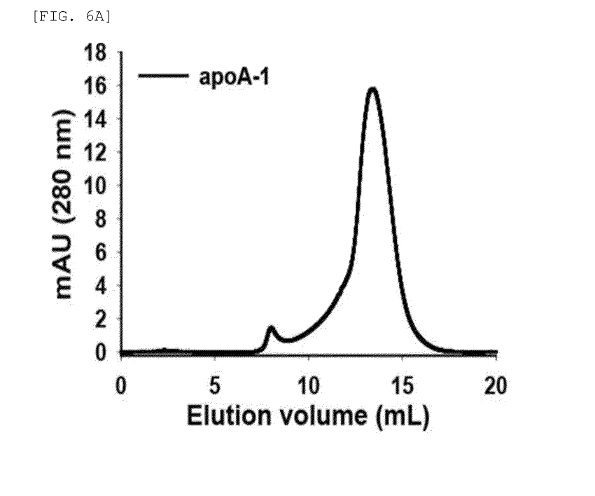

FIG. 6a shows the results of size exclusion chromatography analysis of free apoA-I, and FIG. 6b shows the results of size exclusion chromatography analysis of the composite prepared according to Example 3-1. The peak of the composite prepared according to Example 3-1 appeared at an elution volume corresponding to the peak of free apoA-I, indicating that EpoB was not encapsulated into apoA-I under the above condition.

### EXAMPLE 3-2

### CONDITION 2

A solution containing 1 mg of EpoB and 1 mL of 100 mM sodium cholate was sonicated to encapsulate EpoB in micelles formed of sodium cholate. The sonication was performed at 1-second intervals for a total of 1 minute with a minimum output of 20% or less at low temperature in such a manner that no heat generation occurred.

Then, 1 mL of phosphate buffer containing apoA-I was added to the sonicated solution containing EpoB micelles, and the mixture solution was subjected to sonication three times at 1-second intervals for 30 seconds with an output of 25%.

Next, the mixture solution was diluted with phosphate buffer so that the concentration of sodium cholate was lowered below its critical micelle concentration while apoA-I was mixed with EpoB micelles. Herein, the mixing ratio of apoA-I to EpoB was 1:30 (molar ratio).

Next, a surfactant adsorbent was added to the diluted mixture solution to completely remove sodium cholate present at the critical micelle concentration, and then the mixture solution was subjected sequentially to centrifugation and size exclusion chromatography, thereby preparing an EpoB/apoA-I composite.

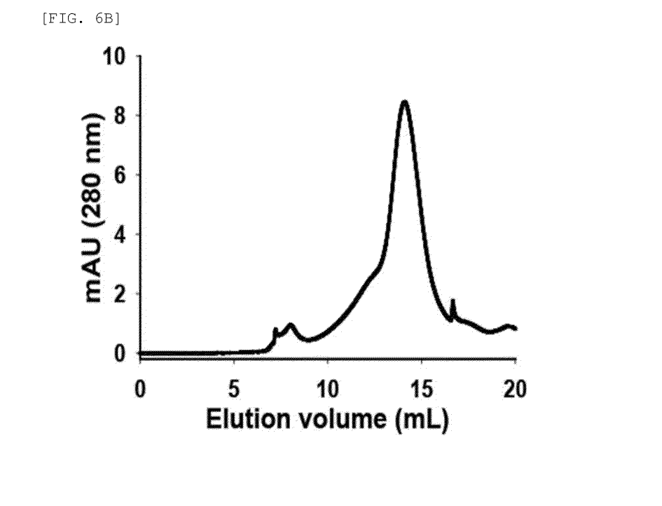

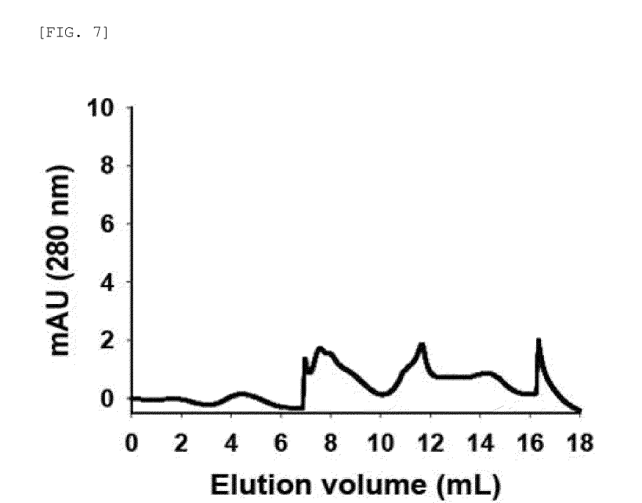

FIG. 7 shows the results of size exclusion chromatography analysis of the composite prepared according to Example 3-2. In Example 3-2, it was attempted to encapsulate EpoB by sonication after mixing EpoB micelles with apoA-I, but excessive heat caused by sonication resulted in the degradation and denaturation of the protein. Thus, it was shown that EpoB was not encapsulated.

### EXAMPLE 3-3

### Condition 3 (Change in Removal Rate of Sodium Cholate)

It was hypothesized that sodium cholate should be completely removed to induce the self-assembly of an EpoB/apoA-I composite. To confirm this hypothesis, the following experiment was performed.

First, a solution containing 1 mg of EpoB and 1 mL of 20 mM sodium cholate was sonicated to encapsulate EpoB in micelles formed of sodium cholate. The sonication was performed at 1-second intervals for a total of 1 minute with a minimum output of 20% or less at low temperature in such a manner that no heat generation occurred.

Then, the sonicated solution containing EpoB micelles was mixed with 1 mL of phosphate buffer containing apoA-I and diluted so that the mixing ratio of apoA-I to EpoB was 1:30 (molar ratio). Then, the mixture solution was subjected to sonication three times at 5-second intervals for 1 minute with an output of 25% at low temperature in such a manner that no heat generation occurred.

Next, a surfactant adsorbent was added to the mixture solution to completely remove sodium cholate present at the critical micelle concentration or lower. Then, the solution was subjected sequentially to centrifugation and size exclusion chromatography, thereby an EpoB/apoA-I composite (hereinafter referred to as EpoB-rHDL).

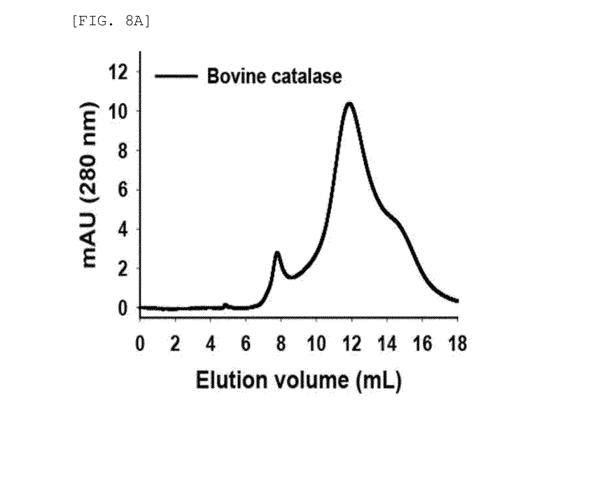

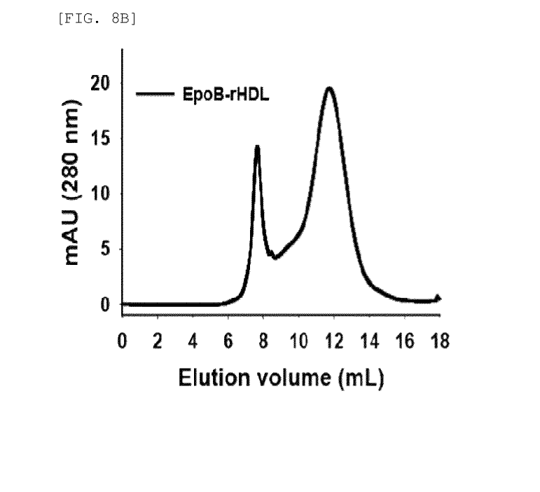

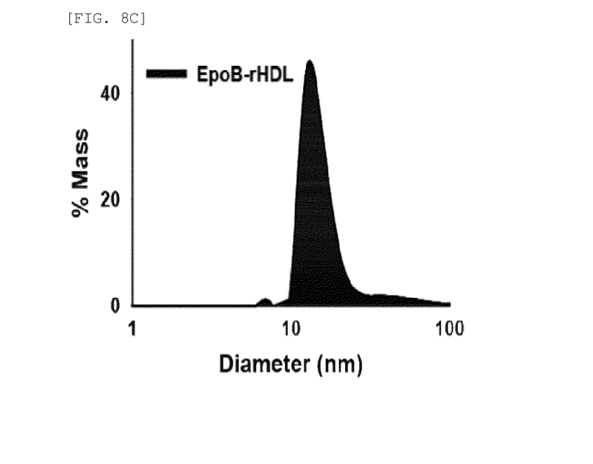

FIGS. 8a to 8c show the determined size of the EpoB-rHDL prepared according to Example 3-3.

The size of the EpoB-rHDL prepared according to Example 3-3 was analyzed by size exclusion chromatography (FIG. 8b), and as a result, the peak at the elution volume peak equal to the peak of bovine catalase having a size of about 10 nm (FIG. 8a) appeared. In addition, the size of the EpoB-rHDL was analyzed by dynamic laser light scattering (FIG. 8c), and as a result, it was determined that a nanoparticle composite having a size of about 10 nm was formed.

However, the analysis of the EpoB-rHDL by HPLC (high performance liquid chromatography) indicated that the yield of the drug in each sample was only 10%.

### EXAMPLE 3-4

### Condition 4 (Change in Conditions in which EpoB is Encapsulated into Micelles)

It was hypothesized that sodium cholate should be completely removed to induce the self-assembly of EpoB-encapsulating apoA-I and a process of encapsulating EpoB into micelles is also important. To confirm this hypothesis, the following experiment was performed.

First, a solution containing 1 mg of EpoB and 1 mL of 100 mM sodium cholate was sonicated to encapsulate EpoB in micelles formed of sodium cholate. The sonication was performed at 1-second intervals for a total of 1 minute with a minimum output of 20% or less at low temperature in such a manner that no heat generation occurred.

Then, the sonicated solution was diluted with phosphate buffer so that the concentration of sodium cholate reached 20 mM, and the diluted solution was subjected to sonication under the same conditions as above (preparation of EpoB-micelles).

Then, the sonicated solution containing EpoB-micelles was mixed with 1 mL of phosphate buffer containing apoA-I. Herein, the mixing ratio of apoA-I to EpoB was 1:30 (molar ratio).

Next, the diluted mixture solution was subjected to sonication three times at 5-second intervals for 1 minute with an output of 25% at low temperature in such a manner that no heat generation occurred. Then, a surfactant adsorbent was added to the mixture solution to completely remove sodium cholate present at the critical micelle concentration or lower. Following this, the solution was subjected sequentially to centrifugation and size exclusion chromatography, thereby preparing an EpoB/apoA-I composite (hereinafter referred to as EpoB-rHDL).

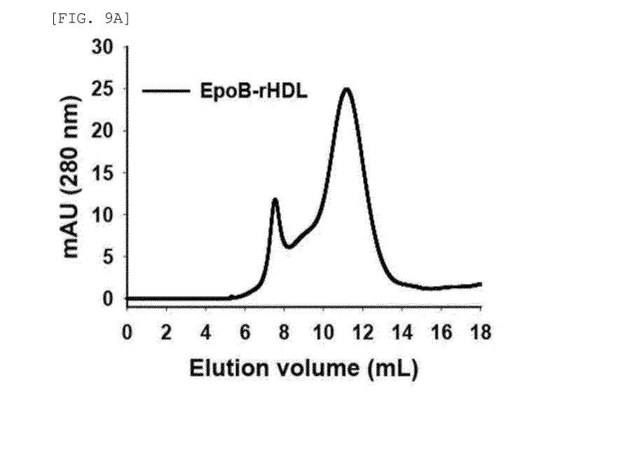

The size of the EpoB-rHDL prepared according to Example 3-4 was analyzed by size exclusion chromatography (FIG. 9a) and dynamic laser light scattering (FIG. 9b), and as a result, it was found that a nanoparticle composite having a size of about 10 nm was formed.

Also, the results of HPLC indicated that the yield of the drug contained in each sample was only 31%.

### EXAMPLE 3-5

### Condition 5

To prepare an EpoB-encapsulating apoA-I without protein denaturation, the following experiment was performed.

First, a solution containing 1 mg of EpoB and 1 mL of 100 mM sodium cholate was sonicated to encapsulate EpoB in micelles formed by sodium cholate. The sonication was performed at 1-second intervals for a total of 1 minute with a minimum output of 20% or less at low temperature in such a manner that no heat generation occurred.

Then, the sonicated solution containing EpoB micelles was mixed with 9 mL of phosphate buffer containing apoA-I and 0.9 M urea, while the concentration of sodium cholate was lowered below its critical micelle concentration. Herein, the mixing ratio of apoA-I to EpoB was 1:30 (molar ratio). Next, the diluted mixture solution was subjected to sonication three times at 5-second intervals for 1 minutes with an output of 25% at low temperature in such a manner that no heat generation occurred.

Thereafter, a surfactant adsorbent was added to the mixture solution to completely remove sodium cholate present at the critical micelle concentration or lower, and then the solution was subjected sequentially to centrifugation and size exclusion chromatography, thereby preparing an EpoB/apoA-I composite (hereinafter referred to as ‘EpoB-rHDL’).

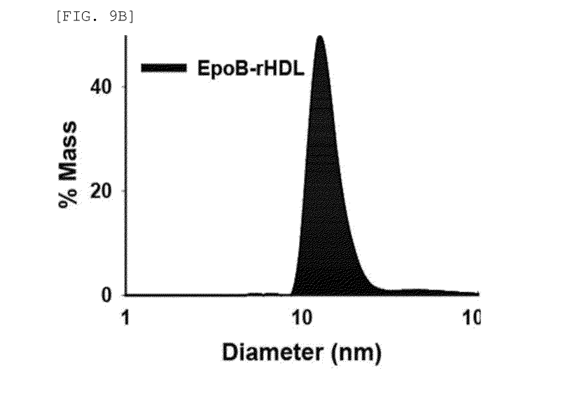

The EpoB-rHDL prepared according to Example 3-5 was analyzed by size exclusion chromatography (FIG. 10a) and dynamic laser light scattering (FIG. 10b), and as a result, it was shown that a nanoparticle composite having a size of about 10 nm was formed.

However, the results of HPLC analysis indicated that the yield of the drug contained in each sample was only 10%.

### EXAMPLE 3-6

### Condition 6 (Change in Mixing Ratio of EpoB to apoA-I)

To increase the efficiency with which EpoB is encapsulated into apoA-I, the mixing ratio of apoA-I to EpoB was changed to 1:20 (molar ratio) to increase the number of moles of apoA-I.

First, a solution containing 1 mg of EpoB and 1 mL of 100 mM sodium cholate was sonicated to encapsulate EpoB in micelles formed by sodium cholate. The sonication was performed at 1-second intervals for a total of 1 minute with a minimum output of 20% or less at low temperature in such a manner that no heat generation occurred.

Then, the sonicated solution containing EpoB micelles was mixed with 9 mL of phosphate buffer containing apoA-I and 0.9 M urea, while the concentration of sodium cholate was lowered below its critical micelle concentration. Herein, the mixing ratio of apoA-I to EpoB was 1:20 (molar ratio). Next, the diluted mixture solution was subjected to sonication three times at 5-second intervals for 1 minutes with an output of 25% at low temperature in such a manner that no heat generation occurred.

Next, a surfactant adsorbent was added to the mixture solution to completely remove sodium cholate present at the critical micelle concentration or lower, and then the solution was subjected sequentially to centrifugation and size exclusion chromatography (SEC), thereby preparing an EpoB/apoA-I composite (hereinafter referred to as EpoB-rHDL).

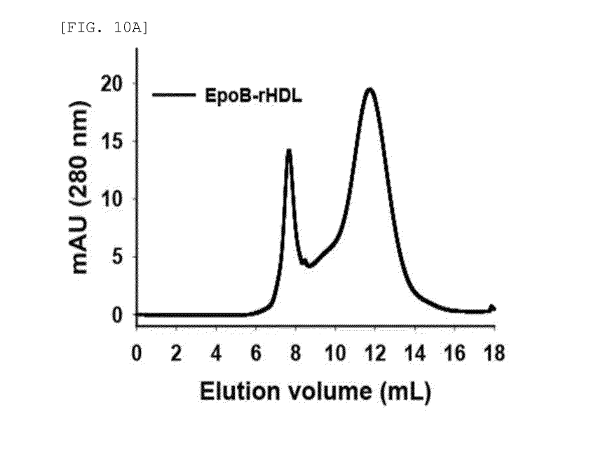

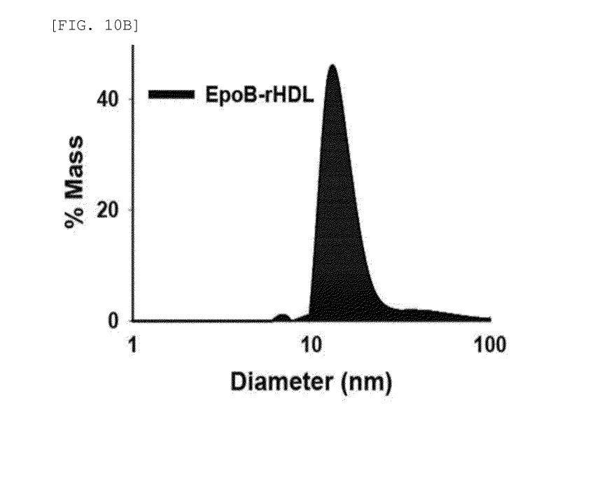

The size of the EpoB-rHDL prepared according to Example 3-6 was analyzed by size exclusion chromatography (FIG. 11a), the peak of the EpoB-rHDL appeared at a lower elution volume compared to the peak of bovine catalase having a size of about 10 nm. Also, the EpoB-rHDL was analyzed by dynamic laser light scattering (FIG. 11b), and as a result, it was shown that a nanoparticle composite having a size of about 20 nm was formed.

### EXAMPLE 4

### Examination of Effect of EpoB-Encapsulating apoA-I

Based on the trial and error of Example 3, methods for preparing an EpoB-encapsulating apoA-I were established.

A first method is a method in which EpoB micelles are formed by sonication in the presence of a surfactant, after which apoA-I is added thereto while diluting the surfactant at a very low rate (Example 4-1).

A second method is a method in which EpoB is dissolved in a solvent, and then EpoB micelles are formed by sonication in the presence of a surfactant, after which apoA-I is added thereto while diluting the surfactant at a very low rate (Example 4-2).

Using the above method, 5 kinds of EpoB-encapsulating apoA-I were prepared. Next, using size exclusion chromatography and reverse-phase chromatography, the initial concentration of EpoB used in the fractionation and preparation of each EpoB-encapsulating apoA-I, and the final concentration and yield of EpoB encapsulated in apoA-I were determined.

### EXAMPLE 4-1

### Preparation of EpoB-Encapsulating apoA-I

A solution containing 1 mg of EpoB and 1 mL of 100 mM sodium cholate was sonicated to encapsulate EpoB in micelles formed of sodium cholate. Herein, the sonication was performed at 5-second intervals for a total of 5 minutes at low temperature with a minimum output of 30% or less using a micro-tip probe sonicator having a size capable of penetrating the sample, in such a manner that no heat generation occurred.

Then, 9 mL of phosphate buffered saline (PBS) containing apoA-I and 0.9 M urea was added dropwise to the sonicated solution containing EpoB-micelles and was diluted so that the concentration of sodium cholate was lowered below its critical micelle concentration while apoA-I was mixed with EpoB micelles. Herein, the mixing ratio of apoA-I to EpoB was 1:30 (molar ratio).

Next, the diluted mixture solution was subjected to sonication at 5-second intervals for 20 minutes with an output of 40% at low temperature in such a manner that no heat generation occurred. Then, the polystyrene copolymer Bio-beads SM-2 (Bio-Rad Laboratories) as a surfactant adsorbent was added to the mixture solution to completely remove sodium cholate present at the critical micelle concentration or lower. Herein, the Bio-beads SM-2 was added to the sample volume in an amount of gram to remove sodium cholate. Thereafter, the solution was subjected sequentially centrifugation and size exclusion chromatography (SEC), thereby preparing an EpoB-encapsulating apoA-I.

### EXAMPLE 4-2

### Change of EpoB Micelle Solution

In order to increase the efficiency with which EpoB is encapsulated in apoA-I, 1 mg of EpoB was dissolved in 100L each of polyethylene glycol (PEG300), 30% PEG300 (diluted with phosphate buffered saline), dimethyl sulfoxide (DMSO) and pure ethanol, which are solvents capable of easily dissolving EpoB. Each of the solutions was sonicated with 900 μL of a solution of 100 mM sodium cholate to encapsulate EpoB in micelles formed by sodium cholate. The sonication process and subsequent processes were performed in the same manner as described in Example 4-1, thereby preparing an EpoB-encapsulating apoA-I.

### EXAMPLE 4-3

### Quantitative Assay of EpoB

EpoB encapsulated in apoA-I was quantified using a high-pressure liquid chromatography (HPLC) apparatus equipped with an XSelect HSS C18 column (Waters, USA) and an ultraviolet detector (HPLC). Pure ethanol and acetonitrile (7:3) were used as an isocratic mobile phase at a flow rate of 1.0 mL/minute. The concentration of EpoB was quantified from a standard curve.

### EXAMPLE 4-4

### Measurement of Final Concentration and Preparation Yield of EpoB Encapsulated in apoA-I

For the EpoB-encapsulating apoA-I prepared according to Example 4-1 or 4-2, the concentration of EpoB dissolved after micelle formation ([EpoB]a) after micelle formation, the micelle formation yield (%), the concentration of EpoB in EpoB-encapsulating apoA-I after removal of the surfactant (final [EpoB]b), the percentage of the concentration of EpoB in the final apoA-I relative to the concentration of EpoB in EpoB micelles in view of dilution of EpoB micelles during preparation (preparation yield c), and the final preparation yield were determined as shown in Table 3 below.

TABLE 3
Final concentration and preparation yield of EpoB encapsulated in apoA-I

| | [EpoB]a | | | | |
|---|---|---|---|---|---|
| | (μg/mL) | | | | Final |
| EpoB | after | Micelle | Final | | prepa- |
| micelle | micelle | formation | [EpoB]b | Preparation | ration |
| solution | formation | yield (%) | (μg/mL) | yield c (%) | yield (%) |
| Sodium | 583 | 58.3 | 52.7 | 90.4 | 52.7 |
| cholate | | | | | |
| 30% | 735 | 73.5 | 67.9 | 92.4 | 67.9 |
| PEG300 + | | | | | |
| sodium | | | | | |
| cholate | | | | | |
| PEG300 + | 743 | 74.3 | 61.4 | 82.6 | 61.4 |
| sodium | | | | | |
| cholate | | | | | |
| DMSO + | 652 | 65.2 | 53.7 | 82.4 | 53.7 |
| sodium | | | | | |
| cholate | | | | | |
| Ethanol + | 684 | 68.4 | 59.4 | 86.8 | 59.4 |
| sodium | | | | | |
| cholate | | | | | |

As shown in Table 3 above, the preparation yield was increased in the method in which EpoB was encapsulated in micelles by sonication in the presence of the surfactant and then the surfactant was diluted at a very low rate while apoA-I was added.

In addition, it was shown that, when EpoB was dissolved in EpoB was dissolved in a solvent capable of easily dissolving EpoB, such as PEG300, DMSO or pure ethanol, before formation of EpoB micelles, and then treated in the same manner as described above, the preparation yield increased by 10%.

### EXAMPLE 4-5

### Size Distribution

EpoB-encapsulating apoA-I was separated by fast protein liquid chromatography (FPLC) using a Superdex-200 10/300 GL column (GE Healthcare, Buckinghamshire, UK). Herein, phosphate buffered saline was used as a mobile phase at a flow rate of 0.5 mL/min. Size distribution was determined by a dynamic laser light scattering technique using a Dynapro apparatus (Wyatt Technology, Santa Barbara, Calif.). The scattering angle and temperature were fixed at 90° and 25° C., respectively.

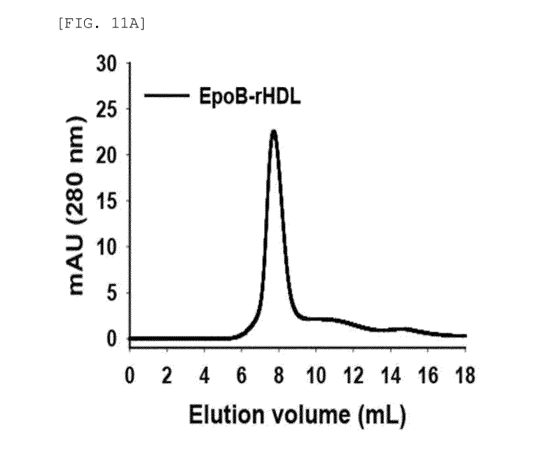

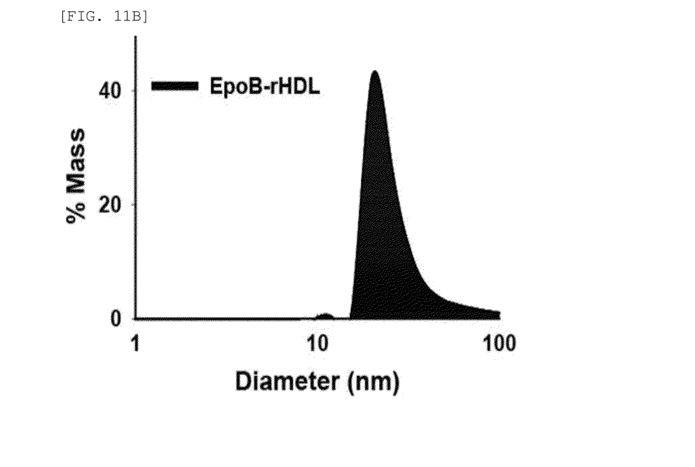

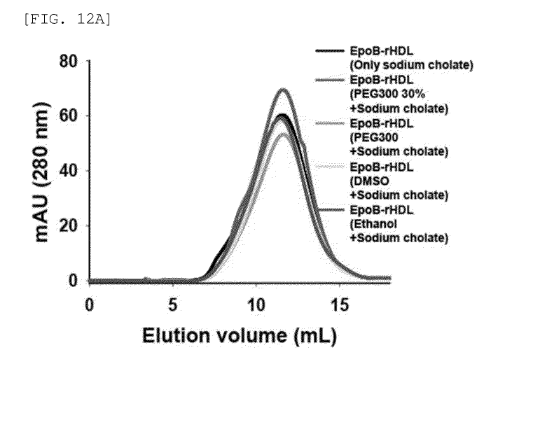

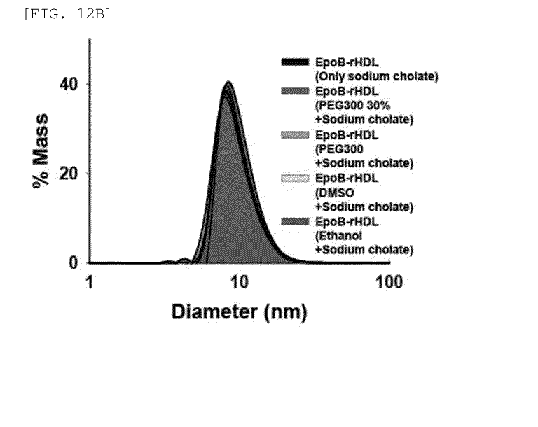

FIGS. 12a to 12d show the determined size of the EpoB-encapsulating apoA-I (hereinafter referred to as EpoB-rHDLs) prepared according to Example 4-1 or 4-2.

The EpoB-rHDLs were analyzed by size exclusion chromatography, and as a result, it was found that, even when different EpoB micelle formation solutions were used, the peaks of the EpoB-rHDLs appeared at the same elution volume and had the same intensity (FIG. 12a).

In addition, the EpoB-rHDLs were subjected to dynamic light scattering analysis, and as a result, it was found that the EpoB-rHDLs prepared using different EpoB micelle formation solutions had the same diameter of about 10 nm (FIG. 12b).

Further, in order to examine the influence of the mixing ratio of apoA-I to EpoB on the efficiency with which EpoB is encapsulated in apoA-I, EpoB-rHDLs prepared while changing the mixing ratio of apoA-I to EpoB to 1:50, 1:30 or 1:20 were subjected to size exclusion chromatography (FIG. 12c) and dynamic light scattering analysis (FIG. 12d).

The results of size exclusion chromatography analysis indicated that the peak of the EpoB-rHDL prepared at a mixing ratio of 1:20 appeared at an elution volume smaller than that of the EpoB-rHDL prepared at a mixing ratio of 1:30 and that the EpoB-rHDL prepared at a mixing ratio of 1:50 showed the peak of free apoA-I together with the peak of EpoB-rHDL, indicating that the efficiency of encapsulation of EpoB was poor (FIG. 12c).

The results of dynamic light scattering analysis indicated that the size of the EpoB-rHDL prepared at a mixing ratio of 1:50 or 1:30 was about 10 nm, whereas the size of the EpoB-rHDL prepared at a mixing ratio of 1:20 was about 18 nm (FIG. 12d).

### EXAMPLE 4-6

### Examination of pH-Dependent Release of Encapsulated EpoB from apoA-I

For cancer chemotherapy, the EpoB-encapsulating apoA-I should release the drug selectively at low pH, and the drug should be stably retained inside the composite at physiological pH.

Because the size of free apoA-I is much smaller than that of the EpoB-encapsulating apoA-I, release of EpoB from the EpoB-encapsulating apoA-I can be easily detected based on a lowered static light scattering. Light scattering intensity is proportional to the amount of the EpoB-encapsulating apoA-I, and thus static light scattering of the prepared EpoB-encapsulating apoA-I was measured for 1 hour at pH 3, 4, 5, 6, 8, 9 and 7.

The static light scattering was measured in the same manner as described in Example 3-4.

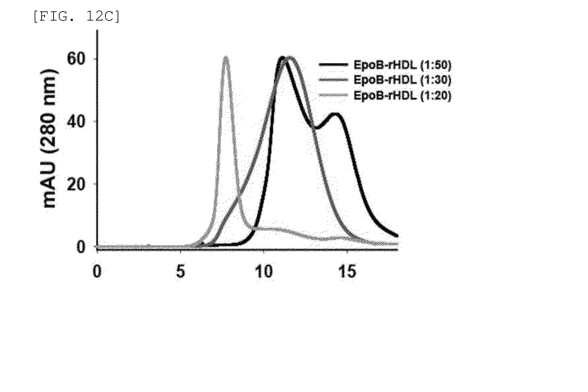

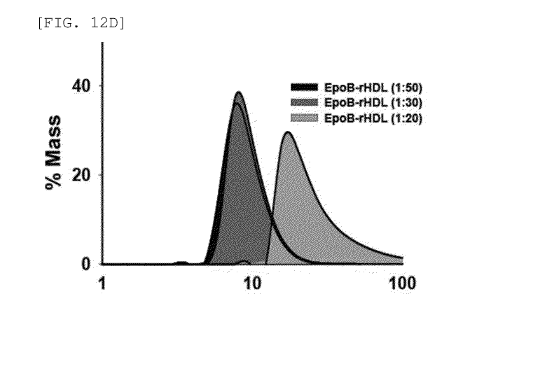

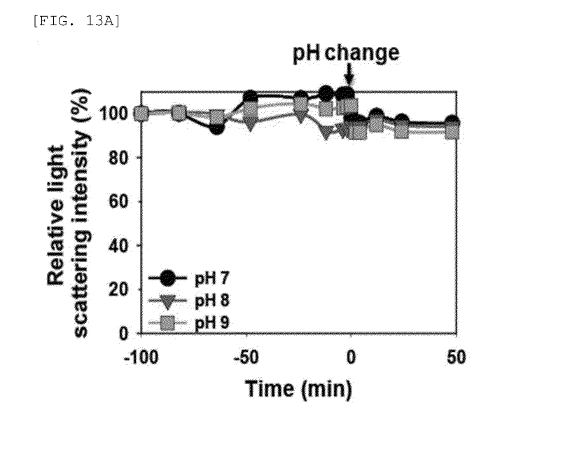

FIGS. 13a and 13b show the time- and pH-dependent release of EpoB from the EpoB-encapsulating apoA-I. Specifically, FIG. 13a shows the results of measuring light scattering intensity, which is proportional to the amount of EpoB-encapsulating apoA-I dissolved, at pH 7, 8 and 9 as a function of time. FIG. 13b shows the results of measuring light scattering intensity, which is proportional to the amount of EpoB-encapsulating apoA-I dissolved, at pH 7, 6, 5, 4 and 3 as a function of time.

As a result, it was shown that, at pH 7 (physiological pH), the light scattering intensity of the EpoB-encapsulating apoA-I did not change. This suggests that the EpoB-encapsulating apoA-I of the present invention is stable at physiological pH. In addition, it was shown that, at pH 8 and 9, the light scattering intensity of the EpoB-encapsulating apoA-I did not change at pH 8 and 9, suggesting that it is stable (FIG. 13a).

In contrast, the light-scattering intensity of the EpoB-encapsulating apoA-I significantly decreased at pH 6 or below, and the rate of decrease in the intensity was more rapid at pH 3 (FIG. 13b). This suggests that the EpoB-encapsulating apoA-I of the present invention can selectively release EpoB at low pH in a pH-dependent manner.

### EXAMPLE 5

### Preparation and Characterization of apoA-I Loaded with Other Drugs

An apoA-I containing the drug doxorubicin (DX), methotrexate (MTX), Sorafenib (SF) or Epothilone B (EpoB) encapsulated therein was prepared, and whether each of the prepared drug-encapsulating apoA-I proteins shows the same characteristics of those prepared in the above Examples.

### EXAMPLE 5-1

### Preparation of apoA-I Loaded with Drugs Other than Paclitaxel

A doxorubicin (DX)-, methotrexate (MTX)-, Sorafenib (SF)- or Epothilone B (EpoB)-encapsulating apoA-I was prepared in the same manner as described in Example 1-3, except that size exclusion chromatography was performed using a Superose-12 10/300 GL column in place of a Superdex-200 10/300 GL column and that the drug DX, MTX, SF or EpoB was used in place of paclitaxel.

### Example 5-2

### Analysis of Size of apoA-I Loaded with Drugs Other than Paclitaxel

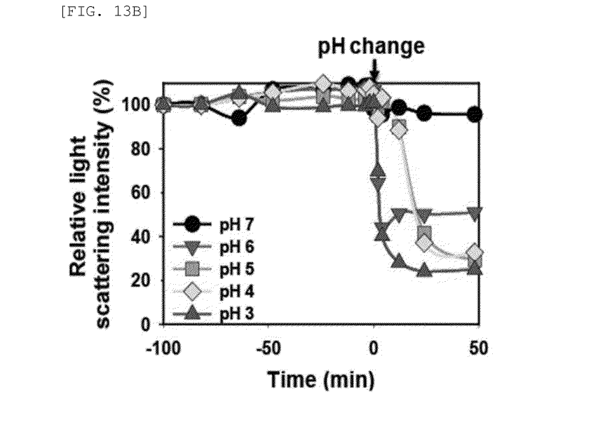

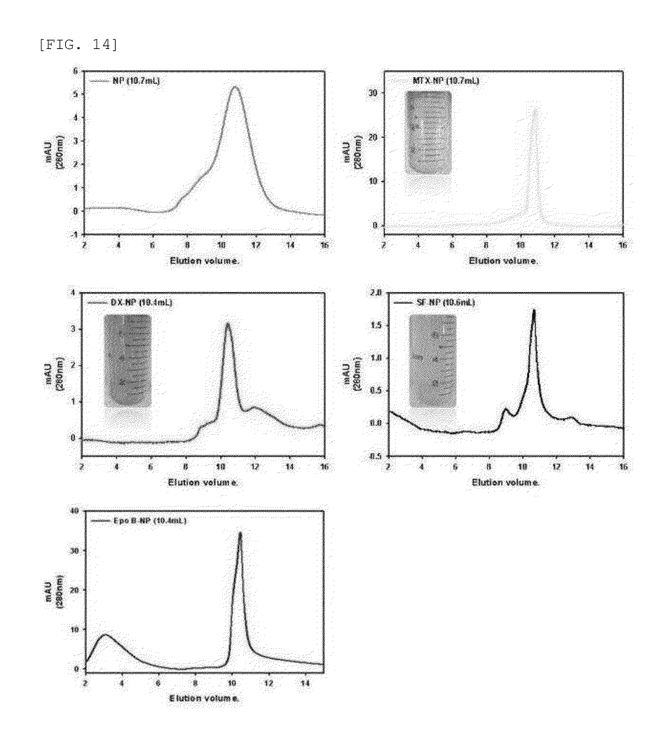

In order to examine whether each of the drug-encapsulating apoA-I proteins prepared in Example 5-1 was normally formed, the size of each of the prepared drug-encapsulating apoA-I proteins was compared with the size of a free apoA-I loaded with no drug (FIG. 14). FIG. 14 is a graphic diagram showing a comparison of size between a free apoA-I and a drug-encapsulating apoA-I prepared by reacting apoA-I with each of DX, MTX, SF or EpoB. As shown in FIG. 14, the apoA-I loaded with each of 4 kinds of different drugs showed the same elution curve as that of the apoA-I loaded with no drug, indicating that the prepared drug-encapsulating apoA-I comprises the drug encapsulated within apoA-I. This suggests that DX, MTX, SF or EpoB is also encapsulated within apoA-I, similar to paclitaxel.

### EXAMPLE 5-3

### Analysis of Whether Drug in Drug-Encapsulating apoA-I was Modified

In order to examine whether the drug in the EpoB-encapsulating apoA-I (Epo-NP) among the drug-encapsulating apoA-I proteins prepared in Example 5-1 was chemically modified by encapsulation, the following experiment was performed.

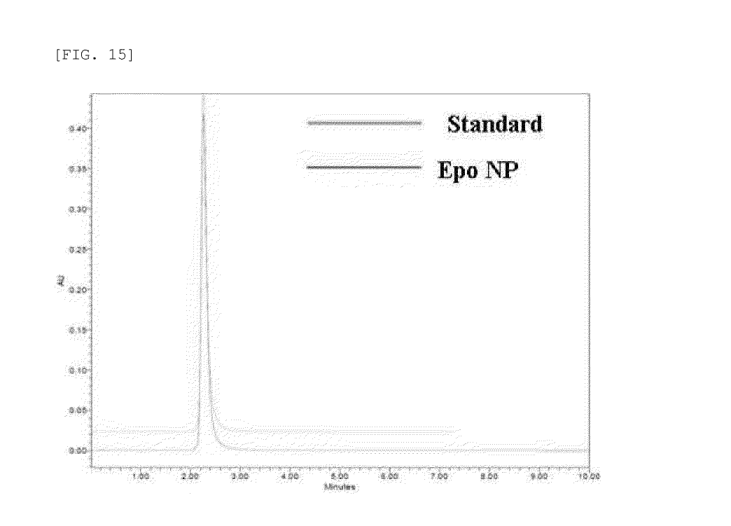

Specifically, to extract the encapsulated drug from the prepared Epo-NP, hexane: 2-propanol (5:3(v/v)) was added to the Epo-NP and allowed to react at 4° C. for about 12 hours. After completion of the reaction, the reaction solution was centrifuged, and the supernatant was recovered. The recovered supernatant was subjected sequentially to nitrogen gas treatment and vacuum treatment to completely remove the solvent. After removal of the solvent, the remaining material was dissolved in methanol, and EpoB extracted from the Epo-NP was analyzed by a high-pressure liquid chromatography (HPLC) apparatus equipped with a Capcell Pak C18 MG reverse phase column (Shiseido, Tokyo, Japan) and an ultraviolet detector, and then the analysis pattern was analyzed to determine whether the extracted EpoB was chemically modified (FIG. 15). In the HPLC analysis, pure methanol was used as a mobile phase at a flow rate of 1.0 mL/minute, and the analysis was performed at a wavelength of 249 nm.

FIG. 15 is a graphic diagram showing a comparison of size between the original EpoB and the EpoB released from Epo-NP. As shown in FIG. 15, EpoB was not chemically modified even when it was encapsulated in apoA-I to form Epo-NP.

### EXAMPLE 5-4

### pH-Dependent Response of EpoB-Encapsulating apoA-I

The results of Example 2-3 indicated that PTX-NP can selectively release PTX at low pH (a pH-dependent release ability), but PTX-PL-NP shows no pH-dependent release ability. Thus, whether Epo-NP comprising EpoB has the same pH-dependent release ability was examined. To examine the pH-dependent release ability, a static light scattering technique was used. In this technique, when EpoB is released from Epo-NP, the static light scattering intensity thereof is proportional to the concentration of EpoB released.

Specifically, Epo-NP was exposed to pH 3, 4, 5, 6, 7, 8 or 10, and the relative scattering intensity of Epo B released from the Epo-NP was measured using a static light scattering technique and compared between pHs (FIG. 16). Herein, the static light scattering technique was performed using a Molecular Device SpectraMax M2 Fluorimeter at an excitation and emission wavelength of 500 nm. Specifically, scattering intensity was measured at pH 7.4 for about 2 hours, and the measured value was set as a standard value. Next, scattering intensity was measured again after increasing or decreasing the pH of the sample, and the pH-dependent release ability of the sample was evaluated based on a change in the scattering intensity.

FIG. 16 is a graphic diagram showing the relative scattering intensity of EpoB-containing Epo-NP at different pHs. As shown in FIG. 16, at pH 7 to 10, the scattering intensity of Epo-NP did not decrease, but at pH 3 to 6, the scattering intensity of Epo-NP decreased, suggesting that EpoB is released from Epo-NP at pH 3 to 6 and that Epo-NP having this property can be used as a suitable therapeutic agent for treatment of cancer cells in vivo, which show acidic pH.

### EXAMPLE 5-5

### Evaluation of Safety of Drug-Encapsulating apoA-I

In order to examine whether a drug-encapsulating apoA-I shows high cell safety compared to the free drug, PTX-NP (that is a paclitaxel-encapsulating apoA-I) and Epo-NP (i.e., an EpoB-encapsulating apoA-I) were prepared. Ovarian SK-OV3 cells, which overexpress HER2 and express a low level of Sr-BI receptor, were treated with varying concentrations (0 to 100 nM) of each of the composites or the drugs, the change in cell viability with a change in the concentration of the composite or the drug was measured.

Specifically, SK-OV3 cells were primarily cultured in RPMI 1640 medium supplemented with 2.05 mM L-glutamine, 10% FBS and antibiotic antimycotic solution (Hyclone), and the cultured cells were counted. Then, the cells were seeded into each well of a 96-well cell culture plate (SPL) at a density of 4×10⁴ cells and incubated in a 5% CO₂ incubator MCO-18AIC (Sanyo Elecric Co.) at 37° C. for 24 hours.

As a negative control for the drug-conjugated apoA-I, a sample obtained by treating cells with PBS (8 g NaCl, 0.2 g KCl, 1.44 g Na₂HPO₄ and 0.24 g KH₂PO₄, pH 7.4); as a negative control for the drug, a sample obtained by treating cells with DMSO (dimethyl sulfoxide); as a positive control indicating the value when cells were completely dead, a sample obtained by treating the cultured cells with 2% Triton X-100 for 2 hours was used; and as a blank, a sample obtained by seeding only medium without cells into plate wells was used.

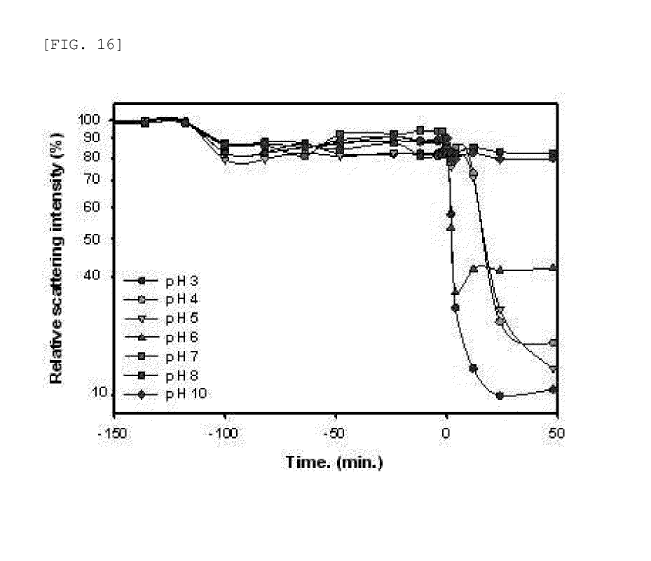

As described above, in order to examine the side effect of the drug or the drug-encapsulating apoA-I, the cultured cells were treated with the drug or the drug-encapsulating apoA-I at a concentration of 0 nM, 1 nM, 2.5 nM, 5 nM, 10 nM, 50 nM or 100 nM, and the viability of the cells treated with the drug or the drug-encapsulating apoA-I was measured by an MTT assay. The measured value was compared to the cell viability obtained by performing an MTT assay for the sample of each control group (FIG. 17).

The MTT assay was performed in the following manner: First, an MTT concentrate obtained by dissolving 3-(4,5-dimethyl-2-thiazolyl)-2,5-diphenyl-2H-tetrazolium bromide (MTT, Calbiochem) in PBS at a concentration of 5 mg/mL was filtered and diluted (10%) to prepare an MTT solution. Meanwhile, the medium and the drug were removed from the cells treated with the drug or the drug-encapsulating apoA-I, and then 100 μL of the prepared MIT solution was seeded into each well and allowed to react at 37° C. in 5% CO₂ for 3 hours. After completion of the reaction, the medium and the MTT solution were removed, and 100 μL of 100% DMSO was seeded into each well and allowed to react at room temperature for 20 minutes under a light-shielded condition. After completion of the secondary reaction, the absorbance of the sample at 570 nm was measured using a spectrophotometer (Spectra M2, Molecular device), and the measured value was substituted into the following equation to determine cell viability:

Cell viability (%)=(S−P−B)/(N−P−B)×100

wherein

S: absorbance of sample treated with drug or drug-encapsulating apoA-I;

B: absorbance of untreated sample;

P: absorbance of positive control; and

N: absorbance of negative control.

FIG. 17 is a graphic diagram showing the change in viability of SK-OV3 cells treated with the drug or the drug-encapsulating apoA-I with a change in the concentration of the drug or the drug-encapsulating apoA-I.

As shown in FIG. 17, treatment with the drug-encapsulating apoA-I showed a cell viability of 50% or higher even when the drug-encapsulating apoA-I was used at a concentration of 100 nM, whereas treatment with the drug showed a cell viability of 50% or lower even when the drug was used at a concentration of 10 nM, suggesting that the drug-encapsulating apoA-I comprising the drug shows high cell safety.

### EXAMPLE 6

### In Vitro Verification of Cell-Targeting Effectiveness and Safety of EpoB-Encapsulating apoA-I

In order to examine whether the EpoB-encapsulating apoA-I can target SR-B1-overexpressing cancer cells using the biological property of recognizing SR-B1(scavenger receptor class B member 1) receptor, five kinds of cancer cell lines that express different levels of SR-B1 were selected based on a literature search. The five kinds of cancer cell lines selected were the breast cancer cell lines MCF7, MDA-MB-231 and ZR-75-1, the uterine cancer cell line SK-OV-3, and the colorectal cancer line Caco-2.

RNA was extracted from each of the selected five kinds of cancer cell lines, and the expression levels of SR-B1 in the cell lines were analyzed by a reverse transcriptase polymerase chain reaction using a primer pair of 5′-ATG GGC TGC TCC GCC AAA GC-3′ (SEQ ID NO: 3) and 5′-CTA CAG TTT TGC TTC CTG CA-3′ (SEQ ID NO: 4).

Then, the expression levels of SR-B1 were analyzed by a quantitative real-time reverse transcription polymerase chain reaction using a primer pair of 5′-CGG CTC GGA GAG CGA CTA C-3′ (SEQ ID NO: 5) and 5′-GGG CTT ATT CTC CAT CAT CAC C-3′ (SEQ ID NO: 6).

In addition, the cancer cells were treated with free EpoB or the EpoB-encapsulating apoA-I at various EpoB concentrations for 72 hours, and the cell viability for each drug was measured using an MTT (3-(4,5-dimethylthiazol-2-yl)-2,5-diphenyltetrazolium bromide) assay. The MTT assay was performed in the same manner as described in Example 3-5.

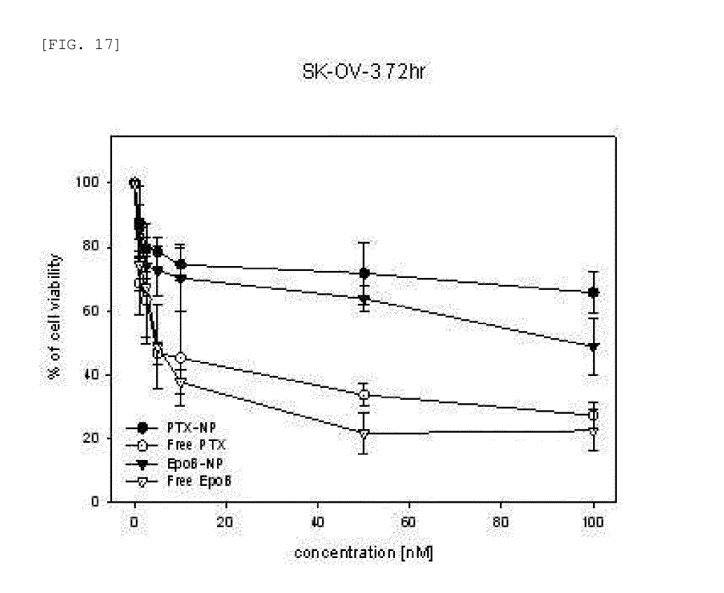

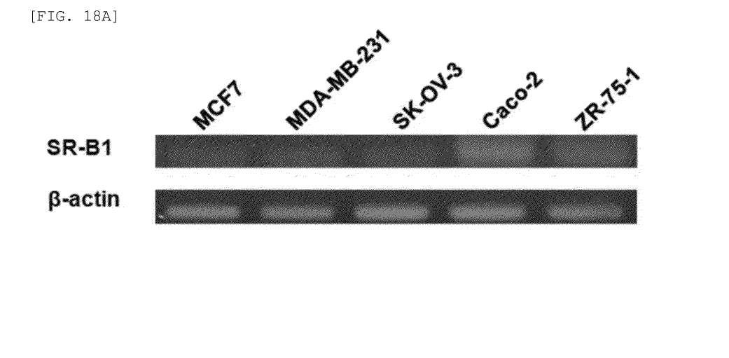

As a result, it was shown that the expression levels of SR-B1 in the MCF7, MDA-MB-231 and SK-OV-3 cell lines were similar to those in normal cells and that the Caco-2 and ZR-75-1 cell lines overexpressed SR-B1 (FIGS. 18a and 18b).

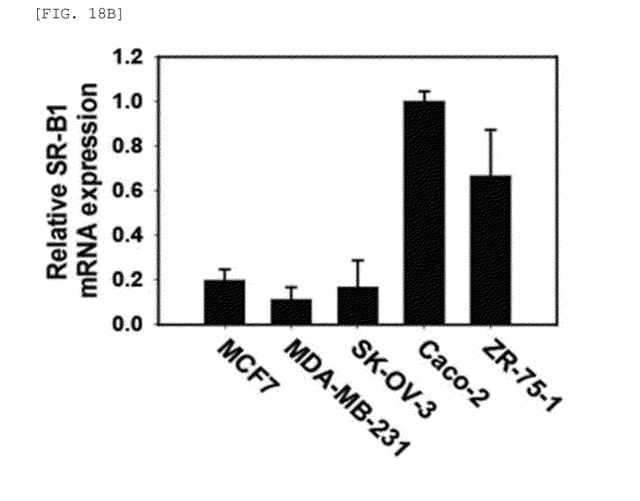

FIG. 19 show the results of an MTT assay for five kinds of cancer cells. Specifically, FIG. 19 shows the cell viabilities of the breast cancer cell line MCF7 (a), the breast cancer cell line MDA-MB-231 (b), the uterine cancer cell line SK-OV-3 (c), the colorectal cancer cell line Caco-2 (d), and the breast cancer cell line ZR-75-1 (e). The five kinds of cancer cell lines showed different expression levels of SR-B1, and thus different cell viabilities. For reference, the concentration of EpoB in the EpoB-encapsulating apoA-I was measured, and EpoB in the EpoB-encapsulating apoA-I was used at a concentration similar to that of free EpoB.

The cancer cell lines MCF7, MDA-MB-231 and SK-OV-3, which do not overexpress SR-B1 (that is recognized by apoA-I) and which express SR-B1 at levels similar to the SR-B1 expression levels of normal cells, showed higher cell viability when they were treated with the EpoB-encapsulating apoA-I compared to when they were treated with free EpoB (FIGS. 19(a), 19(b) and 19(c)).

Meanwhile, the cancer cell lines Caco-2 and ZR-75-1, which overexpress SR-B1, showed higher cell viability when they were treated with free EpoB compared to when they were treated with the EpoB-encapsulating apoA-I (FIGS. 19(d) and 19(e)).

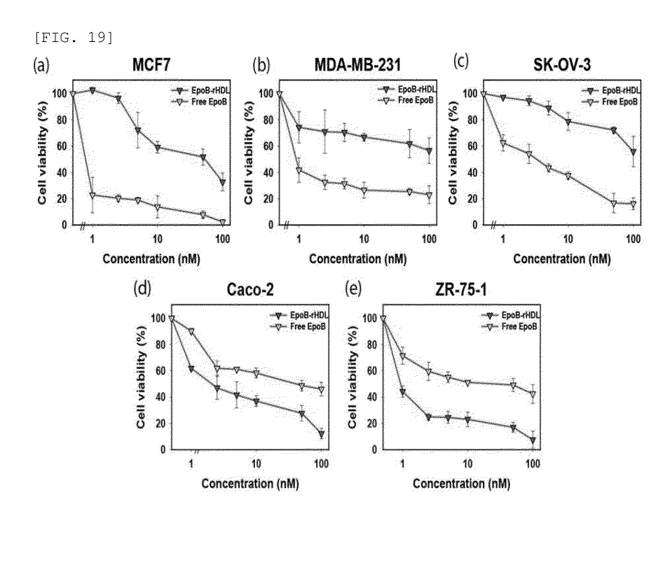

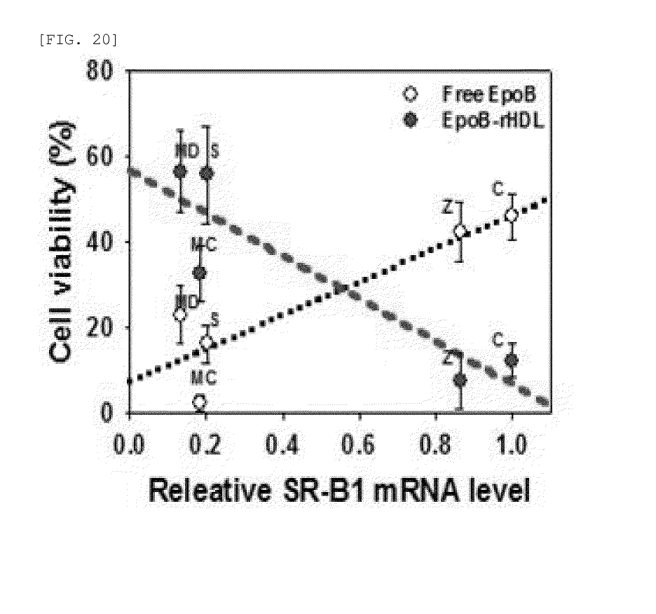

FIG. 20 shows cell viability as a function of the expression level of SR-B1. In FIG. 20, MD represents MDA-MB-231, MC represents MCF7, S represents SK-OV-3, Z represents ZR-75-1, and C represents Caco-2. As can be seen therein, in the case of cells showing higher expression levels of SR-B1, the EpoB encapsulated in apoA-I more effectively induced cell death in cancer cell compared to free EpoB.

The above results indicate that the EpoB-encapsulating apoA-I is a biocompatible protein and can encapsulate a large amount of EpoB to form nanoparticles having a size of about 10 nm. Particularly, it was found that the EpoB-encapsulating apoA-I is pH-dependent so that it is stable at physiological pH and selectively releases the drug at an acidic pH, suggesting its usefulness for cancer chemotherapy.

In addition, it can be concluded that the EpoB-encapsulating apoA-I targets SR-B1, and thus is effective against cancer cell lines that overexpress SR-B1, and the EpoB-encapsulating apoA-I is not toxic to normal cells that do not overexpress SR-B1.

### EXAMPLE 7

### Non-Clinical Study on In Vivo Safety of EpoB-Encapsulating apoA-I

### Example 7-1

### Materials and Method

(1) Test Material

A solution containing the drug Epothilone B (EpoB) was sonicated in the presence of a surfactant (sodium cholate) to encapsulate the EpoB in micelles composed of the surfactant. Then, apoA-I was added to and mixed with the sonicated solution while the concentration of the surfactant was lowered below its critical micelle concentration by dilution. The remaining surfactant was removed by Bio-bead, and the remaining solution was subjected to centrifugation and size exclusion chromatography, thereby preparing an EpoB-encapsulating apoA-I (hereinafter referred to as ‘EpoB-rHDL’).

(2) Control Material

EpoB was dissolved in poly ethylene glycol 300 (PEG 300) and then diluted so that the final concentration of PEG 300 was 30% (v/v). In addition, 30% PEG 300 that is a medium for Epothilone B, and apoA-I were selected as negative controls.

(3) Test Animals

Twenty-eight (seven per group) 6-week-old female ICR mice were purchased from Orientbio Inc. (Korea). The mice were subjected to veterinary inspection for general health conditions and were acclimated to the laboratory environment for 1 week. An animal test was performed in the Laboratory Animal Research Center, Sungkyunkwan University.

(4) Division into Groups

After the acclimation period, the mice were weighed and randomly divided into four groups, each consisting of 7 mice, so that the mice of each group had an average body weight of 24 g.

(5) Individual Identification

An identification card having written test type, test number, test material, individual species, individual number, sex, dosage, test period and test manager was attached to a cage.

(6) Method and Dosage of Administration

The test material was administered into the tail vein of the grouped ICR mice at a dose of 100 μL once a week for 4 weeks. To verify the toxicity and safety of the test material, the concentration of EpoB encapsulated in apoA-I was measured, and the poB-encapsulating apoA-I or the free EpoB was administered at a dose higher than the maximum tolerated dose of EpoB. For the first 2 weeks, the test material was administered to the ICR mice at a dose of 10 mg/kg, which corresponds four times the maximum tolerated dose of EpoB, and for the next 2 weeks, it was administered at a high EpoB dose of 81 mg/kg. The control apoA-I was administered at a dose corresponding to the concentration of apoA-I in the EpoB-encapsulating apoA-I.

(7) Evaluation 1

### body Weight Measurement and Appearance Examination

To observe changes in the body weight of the mice during the test period, the body weight of the mice was measured every day after the start of the test, and diarrhea, hair loss and quadriplegia as the suspected side effects of epoB were observed. When it was observed that the body weight loss exceeded 10% or that the mice showed quadriplegia so as to be difficult to survive, euthanasia, blood collection and autopsy were performed.

(8) Evaluation 2—Serum Chemistry

For liver toxicity analysis, upon the death of the test animals or at the end of the test, the animals were anesthetized with a mixture of Zoletil and Rompun, and then sera were obtained by blood collection from the abdominal aorta. Alanine transferase (ALT) and aspartate transaminase (AST) in the sera were analyzed by an automatic chemistry analyzer.

(9) Evaluation 3—Histopathological Examination

Upon the death of the test animals or at the end of the test, the animals were anesthetized with a mixture of Zoletil and Rompun. Next, the liver was harvested and weighed, and the percentage of the weight of the liver relative to the body weight was comparatively analyzed. In addition, the stomach was harvested and observed to the naked eye.

### EXAMPLE 7-2

### Trial and Error of In Vivo Stability Analysis of EpoB-Encapsulating apoA-I

(1) Trial and Error of Dose of EpoB

EpoB was administered at the maximum tolerated dose that causes no toxicity, once a week for 4 weeks, but toxicity could not be compared between EpoB and the EpoB-encapsulating apoA-I.

Thus, to compare toxicity between EpoB and the EpoB-encapsulating apoA-I, the dose of each of EpoB and the EpoB-encapsulating apoA-I should exceed the maximum tolerated dose that causes no toxicity.

(2) Use of apoA-I Containing Endotoxin

In a primary test, an EpoB-encapsulating apoA-I was prepared using an apoA-I having an endotoxin content of more than 10,000 EU/mg and was administered to ICR mice once a week for 4 weeks. As a result, toxicity was observed not only in the group administered with the endotoxin-containing apoA-I, but also the group administered with the EpoB-encapsulating apoA-I prepared using the apoA-I.

Meanwhile, 22 days after the start of administration of the test material, 4 of 7 animals of the test group administered with the EpoB-encapsulating apoA-I were dead, and thus showed a survival rate of about 46.86%, but the control group administered with alone EpoB showed a survival rate of 100% (FIG. 21). It is believed that the survival rate of the group administered with the EpoB-encapsulating apoA-I was low because of the toxicity of endotoxin of apoA-I in combination with the toxicity of EpoB.

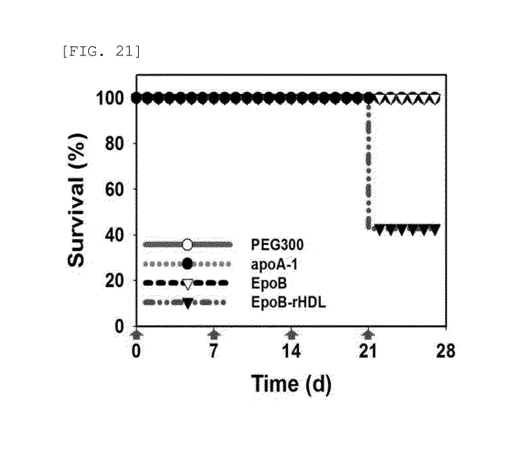

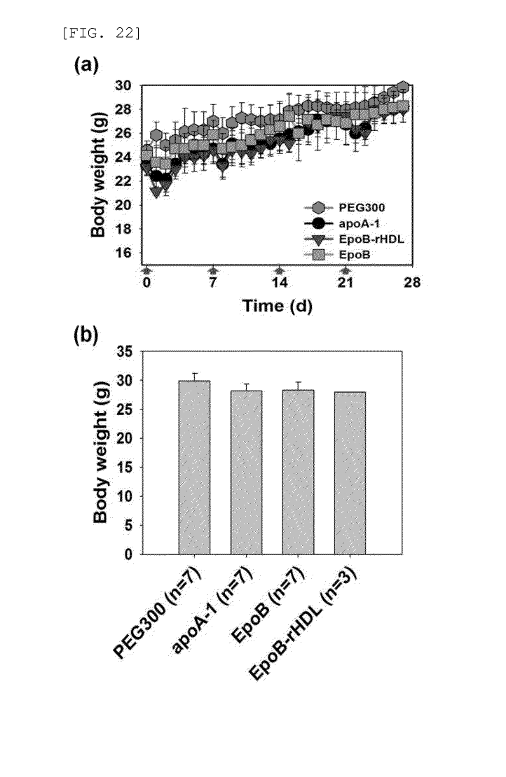

Meanwhile, due to the toxicity of endotoxin of apoA-I, the control group administered with apoA-I showed a higher body weight loss compared to the control group administered with 30% PEG 300, and the control group administered with EpoB also showed a body weight loss due to the toxicity of EpoB (FIG. 22).

After completion of the test, the liver was extracted by autopsy, and the percentage of the weight of the liver relative to the body weight was analyzed. As a result, the EpoB-encapsulating apoA-I showed about an 1.34-fold increase in the liver weight compared to 30% PEG 300 and EpoB and showed 1.26-fold and 1.91-fold increases in AST and ALT, respectively, as analyzed by serum chemistry after blood collection (Table 4).

TABLE 4
Liver toxicities of test materials in ICR mice
administered with test materials

| | Liver (%) | AST (U/L) | ALT (U/L) |
|---|---|---|---|
| PEG300 | 4.6 ± 0.4 | 43.9 ± 5.8 | 22.1 ± 2.9 |
| apoA-I | 6 ± 1.1 | 52.9 ± 13.2 | 34.4 ± 16.9 |
| EpoB | 4.7 ± 0.3 | 45.6 ± 9.3 | 18.1 ± .38 |
| EpoB- | 6.3 ± 0.8 | 54 ± 7.8 | 24.3 ± 3.8 |
| encapsulating | | | |
| apoA-I | | | |

In Table 4 above, the increases in liver weight, AST and ALT (indices of liver toxicity) in the control group administered with endotoxin-containing apoA-I and the test group administered with the EpoB-encapsulating apoA-I prepared using endotoxin-containing apoA-I can appear as a result of non-alcohol fatty livers. This appears that the influence of toxicity of endotoxin is great.

From the above results, it was confirmed that it is important to remove endotoxin from the constituent protein apoA-I of the EpoB-encapsulating apoA-I. Thus, purification of an endotoxin-free apoA-I should be preferentially performed during the experiment.

### EXAMPLE 7-3

### In Vivo Safety Test for EpoB-Encapsulating apoA-I

(1) Endotoxin and Dose Control

Using an apoA-I purified to have an endotoxin content of 5-10 EU/mg, an EpoB-encapsulating apoA-I was prepared according to the method described in Example 7-1(1). The prepared EpoB-encapsulating apoA-I had an endotoxin content of 20-30 EU/mg, and thus is safe for intravenous administration.

EpoB and the EpoB-encapsulating apoA-I were administered at doses of 10 mg/kg and 81 mg/kg, which are higher than the maximum tolerated dose of EpoB.

(2) Examination of Survival Rate, Body Weight Change and Appearance

The dose at week 1 and week 2 was 10 mg/kg, which is four times the maximum tolerated dose of EpoB, but in appearance examination, no adverse effects were observed in the control group administered with EpoB and the test group administered with the EpoB-encapsulating apoA-I. Thus, the dose at week 3 and week 4 was increased to 81 mg/kg, which is a high dose of EpoB.

In the case of the control group administered with EpoB, hair loss occurred after 1 day at week 3. After 3 days at week 3, No. 7 animal showed a body weight loss of about 25% and quadriplegia. After 4 days at week 3, No. 7 animal of the control group administered with EpoB was dead, and Nos. 1, 2, 3 and 5 animals showed a body weight loss of about 7-17% and quadriplegia so as to be difficult to survive, and thus were euthanized. No. 4 animal showed a body weight loss of about 11%, but showed no quadriplegia and could survive, and thus was not euthanized.

On the other hand, in the case of the test group administered with the EpoB-encapsulating apoA-I, high-dose administration of the EpoB-encapsulating apoA-I caused no hair loss, showed a body weight loss of 10% or less, and did not affect the survival of the mice.

In addition, the constituent protein apoA-I of the EpoB-encapsulating apoA-I was also administered at a high dose of about 266 mg/kg, but it did not affect the survival, body weight change and hair loss of the mice.

Meanwhile, in the case of the control group administered with EpoB, after 1 day at week 4 in which the test material was administered at a high dose, No. 4 animal was dead, and after 3 days at week 4, No. 6 animal showed a body weight loss of 11.5%, and after 4 days at week 4, No. 6 animal was dead.

However, in the case of the test group administered with the EpoB-encapsulating apoA-I, the encapsulated EpoB was administered at a high dose, but it did not affect the survival of the mice and did not show hair loss, quadriplegia and a body weight loss of 10% or more, unlike free EpoB showing strong toxicity.

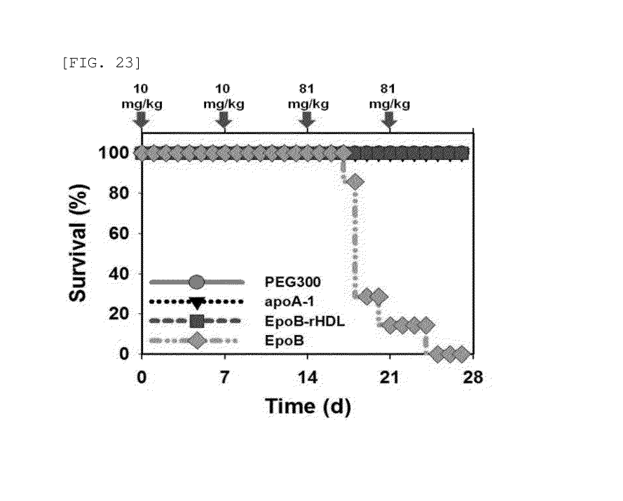

Such results are shown in FIGS. 23 to 29. FIG. 23 shows the survival rates of the control group and the test group, and FIGS. 24 to 29 show changes in the body weights of the control group and the test group.

As shown in FIG. 23, the mice of the group administered with EpoB were dead at week 3 and week 4 due to the high-dose administration of EpoB, but the EpoB-encapsulating apoA-I showed a survival rate of 100%, although it was administered at a high dose.

FIG. 24 shows the body weight of each ICR mouse administered with the test material. As can be seen therein, none of the mice of the test group administered with the EpoB-encapsulating apoA-I showed any significant body weight loss in spite of the high-dose administration of the encapsulated EpoB, unlike the control group administered with EpoB.

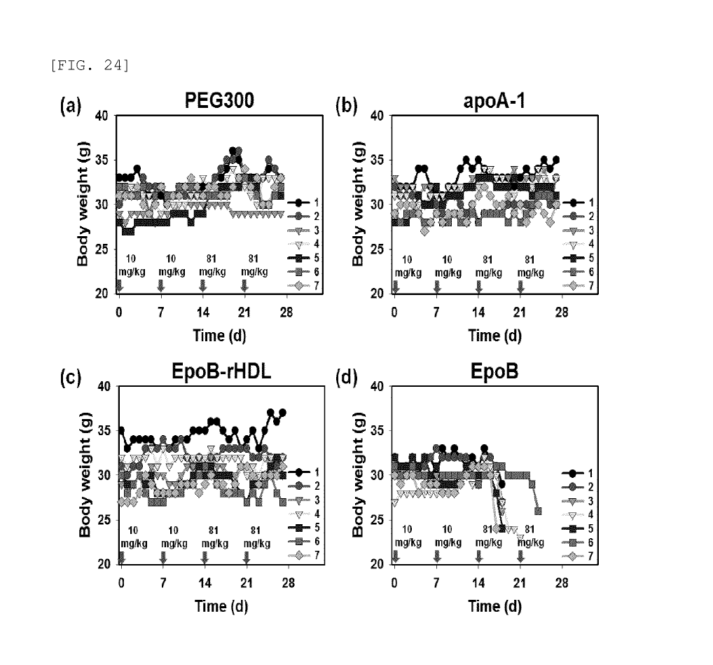

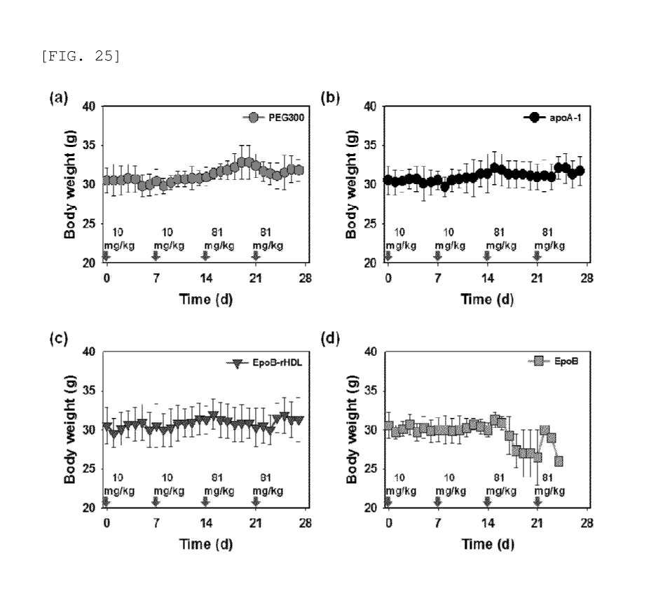

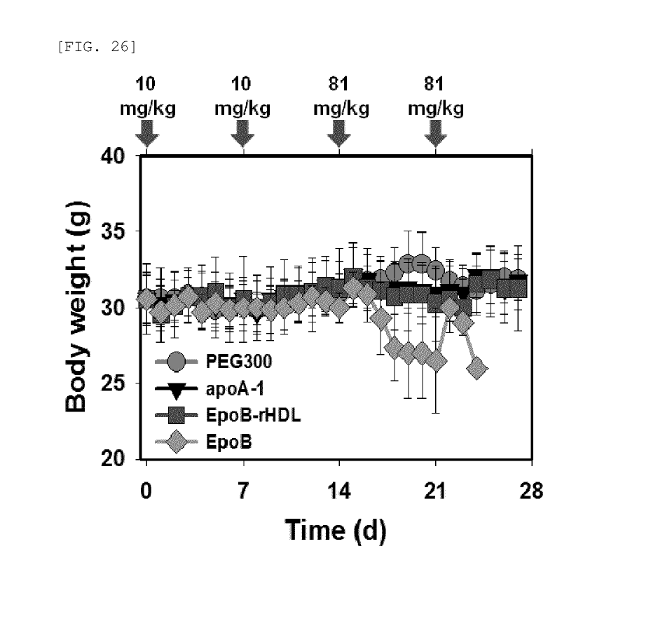

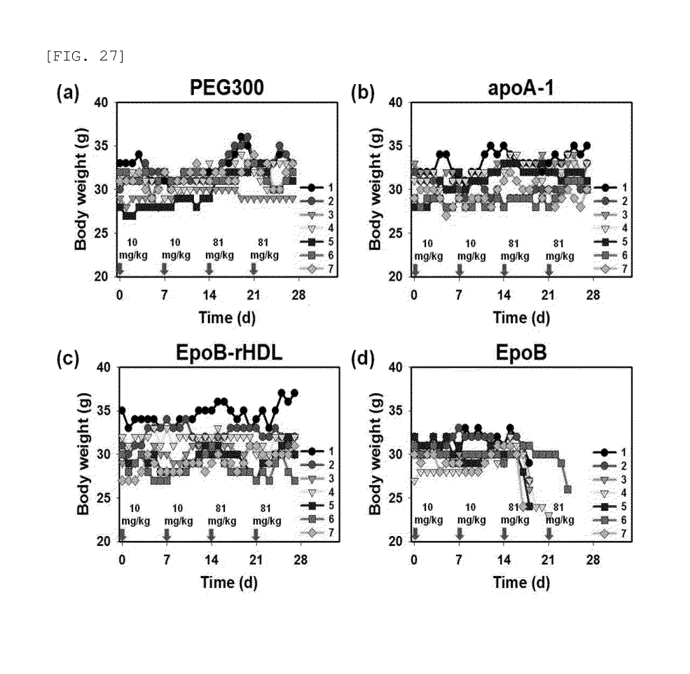

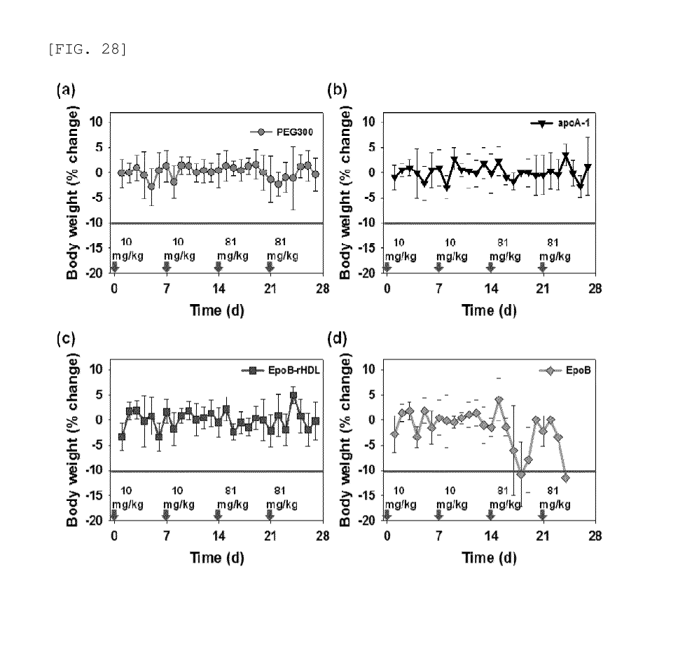

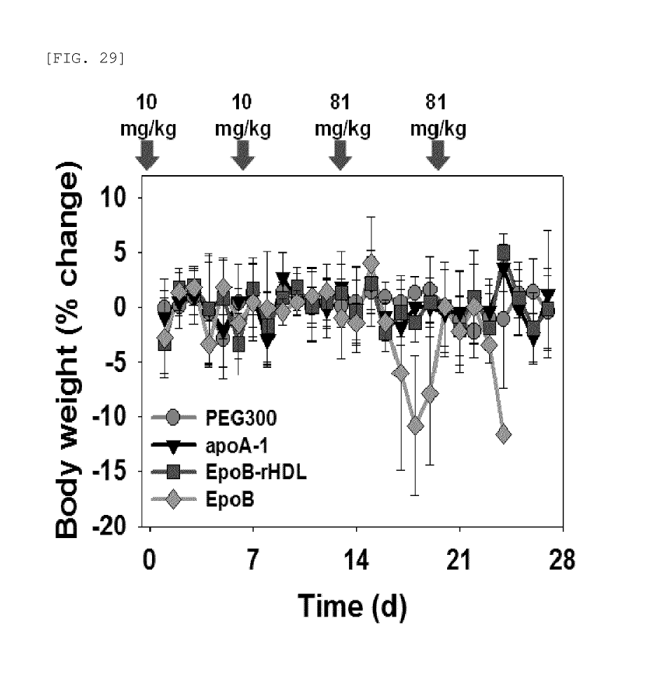

FIG. 25 shows the average body weight of mice administered with each test material; FIG. 26 shows the average body weight of mice administered with each test material; FIG. 27 shows changes in the body weight of each mouse; FIG. 28 shows percent changes in the average body weight of mice administered with each test material; and FIG. 29 shows percent changes in the average body weight of mice. As can be seen therein, none of the mice of the test group administered with the EpoB-encapsulating apoA-I showed a body weight loss of 10% or more in spite of the high-dose administration of the encapsulated EpoB, unlike the control group administered with EpoB.

(3) Liver Toxicity Analysis and Histopathological Examination

Immediately after death or at the end of the test, serum chemistry was performed and the percentage of the weight of the liver relative to the body weight was measured to determine liver toxicity.

The percentage of the weight of the liver relative to the body weight did not significantly differ between the test group administered with the EpoB-encapsulating apoA-I and the control group administered with EpoB. However, the AST value that indicates liver cell damage caused by the drug decreased by about 74% in the test group administered with the EpoB-encapsulating apoA-I compared to that in the control group administered with EpoB, and the ALT value also decreased by about 51% in the test group. In addition, it was shown that the AST/ALT ratio that is used as a standard for determining acute hepatitis among the side effects of EpoB decreased by about 47% in the test group administered with EpoB compared to that in the control group administered with EpoB. Table 5 below shows the liver toxicity of each test material in ICR mice administered with the test materials, and Table 6 below shows the liver toxicity of each test material in each mice.

TABLE 5
Liver toxicity of each test material in ICR mice
administered with test materials

| | Liver (%) | AST (U/L) | ALT (U/L) | AST/ALT |
|---|---|---|---|---|
| PEG300 | 4.27 ± 0.24 | 61.17 ± 10.85 | 21.50 ± 2.63 | 2.92 ± 0.78 |
| apoA-I | 4.12 ± 0.57 | 57.14 ± 11.03 | 21.71 ± 5.03 | 2.70 ± 0.52 |
| EpoB | 4.38 ± 0.28 | 51.83 ± 10.68 | 19.50 ± 7.39 | 2.88 ± 0.78 |
| EpoB- | 4.60 ± 0.9 | 199.03 ± 107.91 | 39.67 ± 24.76 | 5.40 ± 1.54 |
| encap- | | | | |
| sulating | | | | |
| apoA-I | | | | |

In Table 5, above, AST, ALT and AST/ALT values increased because of the activities of AST and ALT due to hemolysis of red blood cells during blood collection. The activities of AST and ALT in red blood cells are 15 times and 7 times those of sera, respectively. Such values were excluded from the average values (** NA: Not Available, *** ND: Not Done).

TABLE 6
Liver toxicity of each test material in each ICR
mouse administered with test material

| | Liver (%) | AST (U/L) | ALT (U/L) | AST/ALTT |
|---|---|---|---|---|
| PEG300 | | | | |
| 1 | 3.82 | 80 | 18 | 4.44 |
| 2 | 4.25 | 55 | 22 | 2.5 |
| 3 | 4.48 | 54 | 18 | 3 |
| 4 | 4.61 | 47 | 25 | 1.88 |
| 5 | 4.1 | 69 | 23 | 3 |
| 6 | 4.31 | >1000* | 210* | NA** |
| 7 | 4.3 | 62 | 23 | 2.7 |
| total | 4.27 ± 0.24 | 61.17 ± 10.85 | 21.50 ± 2.63 | 2.92 ± 0.78 |
| apoA-1 | | | | |
| 1 | 3.83 | 64 | 27 | 2.37 |
| 2 | 3.55 | 73 | 25 | 2.92 |
| 3 | 4.87 | 56 | 29 | 1.93 |
| 4 | 3.42 | 48 | 21 | 2.29 |
| 5 | 4.03 | 69 | 19 | 3.63 |
| 6 | 5 | 41 | 15 | 2.73 |
| 7 | 4.13 | 49 | 16 | 3.06 |
| total | 4.12 ± 0.57 | 57.14 ± 11.03 | 21.71 ± 5.03 | 2.70 ± 0.52 |
| EpoB- | | | | |
| rHDL | | | | |
| 1 | 4.41 | 49 | 22 | 2.23 |
| 2 | 4.09 | 48 | 12 | 4 |
| 3 | 4.33 | 45 | 20 | 2.25 |
| 4 | 4.5 | 43 | 16 | 2.69 |
| 5 | 4.4 | 997* | 154* | 6.47* |
| 6 | 4.93 | 75 | 34 | 2.21 |
| 7 | 3.97 | 51 | 13 | 3.92 |
| total | 4.38 ± 0.28 | 51.83 ± 10.68 | 19.50 ± 7.39 | 2.88 ± 0.78 |
| EpoB | | | | |
| 1 | 3.45 | 159 | 32 | 4.97 |
| 2 | 4.19 | 203 | 31 | 6.55 |
| 3 | 4.85 | 308 | 85 | 3.62 |
| 4 | 6.48 | 365 | 58 | 6.29 |
| 5 | 3.79 | 91 | 12 | 7.58 |
| 6 | 5.12 | 68 | 20 | 3.4 |
| 7 | 4.33 | ND*** | ND*** | ND*** |
| total | 4.60 ± 0.9 | 199.03 ± 107.91 | 39.67 ± 24.76 | 5.40 ± 1.54 |

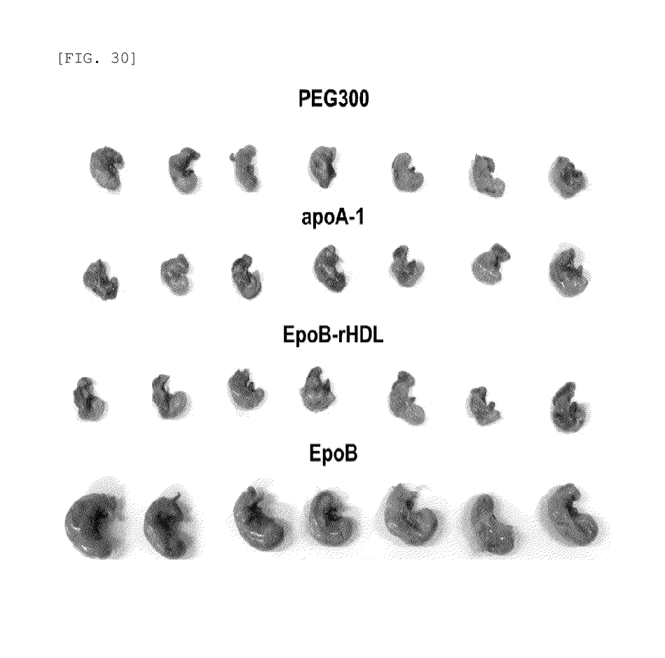

Upon the death of mice or termination of the test, autopsy was performed to observe gastric hypersensitivity caused by abdominal lymphadenopathy, the adverse effect of EpoB. As shown in FIG. 30, the adverse effect of gastric hypersensitivity was observed in the control group administered with EpoB, but not in the test group administered with the EpoB-encapsulating apoA-I.

In conclusion, even when a lethal dose of the EpoB-encapsulating apoA-I was administered to the mice, it did not affect the survival of the mice. Also, the EpoB-encapsulating apoA-I can overcome the adverse effects, including hair loss, quadriplegia symptoms, body weight loss, acute hepatitis and abdominal lymphadenopathy occurring when administered with EpoB. This demonstrates that the EpoB-encapsulating apoA-I is much safer than EpoB.

As described above, the drug-encapsulating apoA-I of the present invention can release the encapsulated drug under acidic conditions, while it is stable under non-acidic conditions over a wide range of temperatures. Thus, it can be widely used for the development of target-specific drugs.

What is claimed is:

1. A drug-encapsulating apoA-I comprising drugs encapsulated in an apoA-I, wherein the drugs are released from apoA-I under acidic pH conditions.

2. The drug-encapsulating apoA-I of claim 1, wherein the apoA-I comprises an amino acid sequence of SEQ ID NO: 1.

3. The drug-encapsulating apoA-I of claim 1, wherein a part of the drug comprises a ring structure.

4. The drug-encapsulating apoA-I of claim 1, wherein the drug is selected from the group consisting of paclitaxel, doxorubicin (DX), methotrexate (MTX), Sorafenib (SF) and Epothilone B (EpoB).

5. The drug-encapsulating apoA-I of claim 1, wherein the drug-encapsulating apoA-I induces cell death in cancer cells that express a high level of SR-B1.

6. A method for preparing the drug-encapsulating apoA-I of claim 1, the method comprising encapsulating drugs in an apoA-I.

7. The method of claim 6, wherein the drug is encapsulated in apoA-I without using a phospholipid or cholesterol.

8. A method for preparing the drug-encapsulating apoA-I of claim 1, the method comprising:
(a) sonicating a buffer comprising drugs and surfactants to encapsulate the drugs into a micelle composed of the surfactant;
(b) mixing the resultant with a buffer comprising apoA-I; and
(c) recovering the drug-encapsulating apoA-I.

9. The method of claim 8, wherein the apoA-I comprises 1-100 EU/mg of endotoxin.

10. The method of claim 8, wherein a part of the drug comprises a ring structure.

11. The method of claim 8, wherein the surfactant forms micelles by sonication.

12. The method of claim 8, wherein the surfactant is sodium cholate.

13. The method of claim 8, wherein the buffer is a disc formation buffer of pH 7.4 containing Tris HCl and NaCl.

14. The method of claim 8, wherein the sonicating is performed at a temperature of from 5 to 15° C.

15. The method of claim 8, wherein the mixing is performed so that a mixing ratio of apoA-I to the drug is maintained at 1:10 to 1:100 (molar ratio) and a concentration of the surfactant is lowered below its critical micelle concentration.

16. The method of claim 8, further comprising, after step (b), a step of completely removing the surfactant.

17. The method of claim 16, wherein the removing of the surfactant is performed using a surfactant adsorbent.

18. The method of claim 17, wherein the surfactant adsorbent is a polystyrene copolymer.

19. The method of claim 8, further comprising a step of removing a precipitate by centrifugation before recovering the drug-encapsulating apoA-I.

20. The method of claim 8, wherein the recovering of the drug-encapsulating apoA-I is performed by size exclusion chromatography.


SEQUENCE LISTING (6 sequences)

SEQ ID NO: 1 - protein, 258 aa, Unknown
  ApoA-I
MHHHHHHGLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - DNA, 777 bp, Unknown
  ApoA-I
atgcatcaccatcaccatcacggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 3 - DNA, 20 bp, Artificial Sequence
  primer
atgggctgctccgccaaagc

SEQ ID NO: 4 - DNA, 20 bp, Artificial Sequence
  primer
ctacagttttgcttcctgca

SEQ ID NO: 5 - DNA, 19 bp, Artificial Sequence
  primer
cggctcggagagcgactac

SEQ ID NO: 6 - DNA, 22 bp, Artificial Sequence
  primer
gggcttattctccatcatcacc